(19) United States
(12) Patent Application Publication
(10) Pub. No.: US 2017/0100332 A1
(43) Pub. Date: Apr. 13, 2017

(54) SURFACE TOPOGRAPHIES FOR NON-TOXIC BIOADHESION CONTROL

(71) Applicants: Sharklet Technologies, Inc., Aurora, CO (US); University of Florida Research Foundation, Inc., Gainesville, FL (US)

(72) Inventors: Leah Tonkin, Memphis, TN (US); Anthony B. Brennan, Gainesville, FL (US); Shravanthi T. Reddy, Goleta, CA (US); Chelsea Marie Magin, Denver, CO (US); Ethan Mann, Centennial, CO (US); Mark Spiecker, Centennial, CO (US); Bryce Stevenson, Centennial, CO (US)

(21) Appl. No.: 15/291,441

(22) Filed: Oct. 12, 2016

Related U.S. Application Data
(60) Provisional application No. 62/240,073, filed on Oct. 12, 2015.

(51) Int. Cl. A61K 9/00; A61K 33/34; A61K 31/567; A61K 31/57; C09D 5/16; A61K 47/34

(52) U.S. Cl. CPC A61K 9/0039; C09D 5/1681; A61K 47/34; A61K 31/567; A61K 31/57; A61K 33/34

(57) ABSTRACT

Disclosed herein is an article that includes a first plurality of spaced features. The spaced features are arranged in a plurality of groupings; the groupings of features include repeat units; the spaced features within a grouping are spaced apart at an average distance of about 1 nanometer to about 500 micrometers; each feature having a surface that is substantially parallel to a surface on a neighboring feature; each feature being separated from its neighboring feature; the groupings of features being arranged with respect to one another so as to define a tortuous pathway. The plurality of spaced features provide the article with an engineered roughness index of about 5 to about 20.

### CROSS REFERENCE TO RELATED APPLICATIONS

This US Non-Provisional application claims the benefit of U.S. Provisional Application Ser. No. 62/240,073, filed Oct. 12, 2015, the entire contents of which are hereby incorporated by reference.

### FIELD OF THE INVENTION

The invention relates to articles and related devices and systems having surface topography and/or surface elastic properties for providing non-toxic bioadhesion control.

### BACKGROUND

Biofouling is the unwanted accumulation of organic and inorganic matter of biological origin on surfaces. For example, in the marine and damp environments biofouling is the result of organisms settling, attaching, and growing on submerged surfaces. The biofouling process is initiated within minutes of a surface being submerged in a damp environment by the absorption of dissolved organic materials which result in the formation of a conditioning film. Once the conditioning film is deposited, bacteria (e.g. unicellular algae) colonize the surface within hours of submersion. The resulting biofilm produced from the colonization of the bacteria is referred to as microfouling or slime and can reach thicknesses on the order of 500 μm.

Any substrate in regular contact with an aqueous or bodily fluid is likely to become fouled. No surface has been found that is completely resistant to fouling. Due to the vast variety of organisms that form biofilms, the development of a single surface coating with fixed surface properties for the prevention biofilm formation for all relevant marine organisms is a difficult if not impossible task.

Anti-fouling and foul-release coatings are two main approaches currently used for combating biofilm formation. Anti-fouling coatings prevent or deter the settling of biofouling organisms on a surface by the use of leached biocides, typically cuprous oxide or tributyltin, into the water. The biocides are either tethered to the coated surface or are released from the surface into the surrounding environment. Use of these types of coatings has caused damage to the marine ecosystem, especially in shallow bays and harbors, where the biocides can accumulate. As such, the use of tributyltin has been banned in many parts of the world. These products are effective for only approximately 2 to 5 years.

Foul release coatings present a hydrophobic, low surface energy, and resulting slippery surface that minimizes the adhesion of the biofouling organisms. The most commonly used and highly successful of these is a nontoxic silicone-based paint. The silicone-based coating requires several layers to make it effective, and therefore it can be quite costly. Effectiveness lasts up to 5 years at which time recoating may become necessary. These products are considered to be more environmentally sound as compared to anti-fouling coatings because they do not leach toxins. However, they are subject to abrasion, and therefore their use is limited to areas that are not susceptible to damage caused by ice or debris.

Biofouling is similarly a problem for surfaces used in biomedical applications. The accumulations of bacteria, i.e. a biofilm, on implanted devices such as orthopedic prostheses present a significant risk of infection leading to complications as severe as death. In cosmetic implants, devices such as breast implants are fouled with fibroblasts and acellular extracellular matrix resulting in a hard fibrous capsule and subsequent implant rupture. Blood contacting surfaces such as artificial heart valves and artificial vascular grafts are fouled by proteins such as fibrinogen that initiate the coagulation cascade leading in part to heart attack and stroke. The accumulated affect of biofouling on chronic and acute disease states, its contribution to morbidity and its massive medical expenses places biofouling as one of the major issues facing modern medicine.

### SUMMARY OF THE INVENTION

An article has a surface topography for resisting bioadhesion of organisms and includes a base article having a surface. The chemical composition of the surface comprises a polymer. The surface has a topography comprising a pattern defined by a plurality of spaced apart features attached to or projected into the base article. The plurality of features each have at least one microscale dimension and at least one neighboring feature having a substantially different geometry. An average spacing between adjacent ones of the features is between 1 μm and 100 μm in at least a portion of the surface.

Surface topographies according to the invention resist bioadhesion as compared to the base article. As used herein, a surface that provides a surface topography according to the invention can be applied to a surface as either a printed patterned, adhesive coating containing the topography, or applied directly to the surface of the device through micromolding. In the case of micromolding, the surface topography will be monolithically integrated with the underlying article.

The feature spacing distance as used herein refers to the distance between adjacent features. Moreover, as used herein, “microscale features” includes micron size or smaller features, thus including microscale and nanoscale.

In one embodiment of the invention referred to as a hierarchical architecture, at least one multi-element plateau layer is disposed on a portion of the surface. A spacing distance between elements of the plateau layer provides a second feature spacing being substantially different as compared to the first feature spacing. The hierarchical architecture can simultaneously repel organisms having substantial different sizes, such as spores and barnacles. In one embodiment the surface is monolithically integrated with the base article, wherein a composition of the base article is the same as the composition of the surface. In another embodiment, the surface comprises a coating layer disposed on the base article. In this coating embodiment, the composition of the coating layer is different as compared to a composition of the base article, and the polymer can comprise a non-electrically conductive polymer, such as selected from elastomers, rubbers, polyurethanes and polysulfones.

The topography can provide an average roughness factor (R) of from 4 to 50 and an elastic modulus of between 10 kPa and 10 MPa. In another embodiment, the topography is numerically representable using at least one sinusoidal function, such as two different sinusoidal waves. An example of a two different sinusoidal wave topography comprises a Sharklet topography. In another embodiment, the plurality of spaced apart features can have a substantially planar top surface. In a preferred embodiment for controlling barnacles, the first feature spacing can be between 15 and 60 μm.

In the multi-element plateau layer disposed on a portion of surface embodiment, wherein a spacing distance between elements of the plateau layer provide a second feature spacing being substantially different as compared to the first feature spacing, the surface can comprise a coating layer disposed on the base article. The elastic modulus of the coating layer can be between 10 kPa and 10 MPa.

The base article can comprise a roofing material. In another embodiment, the base article comprises a water pipe, wherein the surface is provided on an inner surface of a water inlet pipe. In this embodiment, the inlet pipe can be within a power plant in another embodiment, the base article comprises an implantable device or material, such as a breast implant, a catheter or a heart valve.

In another embodiment of the invention, an article has a surface topography for resisting bioadhesion of organisms, comprising a base article having a surface. The composition of the surface comprises a polymer, the surface having a topography comprising a pattern defined by a plurality of spaced apart features attached to or projected into the base article. The plurality of features each have at least one microscale dimension and at least one neighboring feature having a substantially different geometry, wherein an average first feature spacing between adjacent ones of the features is microscale and topography is numerically representable using at least one sinusoidal function. The surface can comprise a coating layer disposed on the base article. In a first embodiment the first feature spacing is between 0.5 and 5 μm in at least a portion of the surface, while in a second embodiment the first feature spacing is between 15 and 60 μm in at least a portion of the surface. The at least one sinusoidal function can comprise two different sinusoidal waves, such as a Sharklet topography. The article can further comprise at least one multi-element plateau layer disposed on a portion of the surface, wherein a spacing distance between elements of the plateau layer provide a second feature spacing being substantially different as compared to the first feature spacing.

Disclosed herein is an article comprising a plurality of spaced features; the spaced features arranged in a plurality of groupings; the groupings of features comprising repeat units; the spaced features within a grouping being spaced apart at an average distance of about 1 nanometer to about 500 micrometers; each feature having a surface that is substantially parallel to a surface on a neighboring feature; each feature being separated from its neighboring feature; the groupings of features being arranged with respect to one another so as to define a tortuous pathway.

Disclosed herein too is an article comprising a plurality of spaced features; the features arranged in a plurality of groupings; the groupings of features comprising repeat units; the spaced features within a grouping being spaced apart at an average distance of about 1 nanometer to about 500 micrometers; the groupings of features being arranged with respect to one another so as to define a tortuous pathway and wherein a tangent to the tortuous pathway intersects with a spaced feature; the spaced feature being different from each nearest neighbor and not in contact with the nearest neighbor.

Disclosed herein too is an intrauterine system (IUS) contraceptive device and the associated monofilament that includes the Sharklet® microtopographic patterned surface. In another embodiment, disclosed herein too is an intrauterine system (IUS) contraceptive device and the associated monofilament that includes a microtopographic patterned surface. The microtopograhic surface comprises a plurality of spaced features; the features arranged in a plurality of groupings; the groupings of features comprising repeat units; the spaced features within a grouping being spaced apart at an average distance of about 1 nanometer to about 500 micrometers; the groupings of features being arranged with respect to one another so as to define a tortuous pathway and wherein a tangent to the tortuous pathway intersects with a spaced feature; the spaced feature being different from each nearest neighbor and not in contact with the nearest neighbor. The IUS is a female implant used for contraception and/or cycle control, the function of which will remain unchanged from current IUSs. The micropattern is a surface that inhibits microbial migration and colonization, the inclusion of which is meant to reduce infections caused by use of this device.

### BRIEF DESCRIPTION OF THE DRAWINGS

A fuller understanding of the present invention and the features and benefits thereof will be obtained upon review of the following detailed description together with the accompanying drawings, in which:

FIG. 1A is a scanned SEM image of an exemplary “Sharklet” anti-algae surface topography comprising a plurality of raised surface features which project out from the surface of a base article, according to an embodiment of the invention.

FIG. 1B is a scanned optical profilometry image of a pattern having a plurality of features projecting into the surface of a base article, according to another embodiment of the invention.

FIG. 2A illustrates exemplary surface architectural patterns according to the invention.

FIG. 2B illustrates exemplary surface architectural patterns according to the invention.

FIG. 2C illustrates exemplary surface architectural patterns according to the invention.

FIG. 2D illustrates exemplary surface architectural patterns according to the invention.

FIG. 3 provides a table of exemplary feature depths, feature spacings, feature widths and the resulting roughness factor (R) based on the patterns shown in FIGS. 2(A)-(D).

FIG. 4A is a depiction of an exemplary hierarchical surface topography according to an embodiment of the invention.

FIG. 4B is a depiction of an exemplary hierarchical surface topography according to an embodiment of the invention.

FIG. 5A shows a sinusoidal wave beginning at the centroid of the smallest (shortest) of the four features comprising the Sharklet pattern.

FIG. 5B shows sine and cosine waves describing the periodicity and packing of the Sharklet pattern.

FIG. 6A shows two (of four) exemplary Sharklet elements, element 1 and element 2; the element 1 and element 2 have different lengths and widths.

FIG. 6B shows the resulting layout after following limitations 3 & 4 (described below) and defining XD. PS (y-spacing between smaller element and larger element after packing).

FIG. 7A shows a space filled with elements that have a different periodicity;

FIG. 7B shows the result of applying sinusoidal waves to define aperiodic structures.

FIG. 7C shows the resulting topographical structure over the full area of the desired surface.

FIG. 8A shows settlement density data for algae spores on a smooth control sample as compared to the settlement density on the Sharklet surface architecture according to the invention shown in FIG. 1(A);

FIG. 8B is a scanned light micrograph image showing algae spores on the surface of the control sample.

FIG. 8C is a scanned light micrograph image showing a dramatic reduction in algae spores on the surface of the surface architecture according to the invention shown in FIG. 1A.

FIG. 9 shows a correlation between Ulva Spore settlement density and the corresponding Engineered Roughness Index (ERI) for several topographical patterns according to the invention (shown in A-D above the correlation data).

FIG. 10 shows settlement data from B. amphitrite on various (PDMS elastomer (PDMSe) channel topographies. Mean values ±1 standard error are shown.

FIG. 11A is a chart showing barnacle cyprid settlement for a first assay (assay 1). Cyprids were allowed to settle for 48 hrs on each of the test surfaces. Topographies used included 20×20 channels (20CH), 20×20 Sharklet (20SK), 20×40 channels (40CH) and 20×40 Sharklet (40SK). Error bars represent ±1 standard error.

FIG. 11B is a chart of barnacle cyprid settlement for a second assay (assay 2). Cyprids were allowed to settle for up to 72 hrs on each of the test surfaces. Topographies used included 20×20 channels (20CH), 20×20 Sharklet (20SK), 40×40 channels (40CH) and 40×40 Sharklet (40SK). Error bars represent ±1 standard error.

FIG. 11C is a chart of barnacle cyprid settlement for a third assay (assay 3). Cyprids were allowed to settle for up to 48 hrs on each of the test surfaces. Topographies used included 20×20 channels (20CH), 20×20 Sharklet (20SK), 40×40 channels (40CH) and 40×40 Sharklet (40SK). Error bars represent ±1 standard error.

FIG. 12 is a photograph showing the fractal dimensions measured parallel to the surface of the substrate.

FIG. 13 is a photograph showing a pattern having nanometer sized features.

FIG. 14A depicts how the degrees of freedom are calculated.

FIG. 14B shows a pattern that results in one degree of freedom for an organism traveling along a channel.

FIG. 15 is a bar chart showing bacterial colonization data on the textured surfaces detailed herein.

FIG. 16 is a bar chart that shows how the textured surfaces detailed herein prevent bacterial migration.

FIG. 17 is a schematic side view of the plunger part of an inserter for positioning an intrauterine device, in accordance with an embodiment of the invention.

FIG. 18A is a side view of the sleeve part of an inserter for positioning an intrauterine device, in accordance with an embodiment of the invention.

FIG. 18B is a bottom view of the sleeve part of an inserter for positioning an intrauterine device, in accordance with an embodiment of the invention.

FIG. 19 is a schematic illustration of an intrauterine device in accordance with an embodiment of the invention.

FIG. 20 is a schematic illustration of an intrauterine device in accordance with another embodiment of the invention.

FIG. 21A demonstrates the relative position of the tube, plunger and the during storage.

FIG. 21B demonstrates the relative position of the tube, plunger and the IUD immediately before, insertion.

FIG. 21C demonstrates the relative position of the tube, plunger and the IUD immediately after the IUD is positioned in the uterus and before the inserter in withdrawn.

### DETAILED DESCRIPTION OF THE INVENTION

The present invention describes a variety of scalable surface topographies for modification of biosettlement and bioadhesion, such as bioadhesion of biofouling organisms, including, but not limited to, algae, bacteria and barnacles. As described in the Examples below, it has been proven through experimental testing that surface topographies according to the invention provide a passive and non-toxic surface, which through selection of appropriate feature sizes and spacing, can significantly and generally dramatically reduce settlement and adhesion of the most common fouling marine algae known, as well as the settlement of barnacles. In one embodiment, the plurality of spaced features increase the effectiveness of algaecides or antibiotics. The algaecides and/or antibiotics can be contained in the surface and can be released gradually when desired.

Disclosed herein are articles comprising a plurality of spaced features; the features arranged in a plurality of groupings; the groupings of features being arranged with respect to one another so as to define a tortuous pathway when viewed in a first direction. When viewed in a second direction, the groupings of features are arranged to define a linear pathway.

In one embodiment, when viewed in a second direction, the pathway between the features may be non-linear and non-sinusoidal. In other words, the pathway can be non-linear and aperiodic. In another embodiment, the pathway between the features may be linear but of a varying thickness. The plurality of spaced features may be projected outwards from a surface or projected into the surface. In one embodiment, the plurality of spaced features may have the same chemical composition as the surface. In another embodiment, the plurality of spaced features may have a different chemical composition from the surface.

In one embodiment, an article having a surface topography for resisting bioadhesion of organisms, comprises a base article having a surface. The composition of the surface and/or the base article comprises a polymer, a metal or an alloy, a ceramic. Combinations of polymers, metals and ceramics may also be used in the surface or the base article. The surface having a topography comprising a plurality of patterns; each pattern being defined by a plurality of spaced apart features attached to or projected into the base article. The plurality of features each have at least one microscale (micrometer or nanometer sized) dimension and has at least one neighboring feature having a substantially different geometry. The average first feature spacing between the adjacent features is between 10 μm and 100 μm in at least a portion of the surface, wherein said plurality of spaced apart features are represented by a periodic function. It is to be noted that each of the features of the plurality of features are separated from each other and do not contact one another.

In one embodiment, the surface is monolithically integrated with said base article, wherein a composition of the base article is the same as the composition of the surface. In another embodiment, the surface comprises a coating layer disposed on the base article. In yet another embodiment, the composition of the coating layer is different from the composition of the base article. In one embodiment, the polymer comprises a non-electrically conducting polymer.

In another embodiment, the topography provides an average roughness factor (R) of from 4 to 50. The surface may comprise an elastomer that has an elastic modulus of about 10 kPa to about 10 MPa.

As noted above, the pattern is separated from a neighboring pattern by a tortuous pathway. The tortuous pathway may be represented by a periodic function. The periodic functions may be different for each tortuous pathway. In one embodiment, the patterns can be separated from one another by tortuous pathways that can be represented by two or more periodic functions. The periodic functions may comprise a sinusoidal wave. In an exemplary embodiment, the periodic function may comprise two or more sinusoidal waves.

In another embodiment, when a plurality of different tortuous pathways are represented by a plurality of periodic functions respectively, the respective periodic functions may be separated by a fixed phase difference. In yet another embodiment, when a plurality of different tortuous pathways are represented by a plurality of periodic functions respectively, the respective periodic functions may be separated by a variable phase difference.

In one embodiment, the plurality of spaced apart features have a substantially planar top surface. In another embodiment, a multi-element plateau layer can be disposed on a portion of the surface, wherein a spacing distance between elements of said plateau layer provide a second feature spacing; the second feature spacing being substantially different when compared to the first feature spacing.

In one embodiment, the pattern comprises a coating layer disposed on said base article. In other words, the coating layer comprises the pattern and is disposed on the base article.

In another embodiment, an article having a surface topography for controlling (e.g., resisting or facilitating) the bioadhesion of organisms, comprises a base article having a surface; wherein the composition of the surface comprises a polymer, a ceramic or a metal. The surface has a topography comprising a pattern defined by a plurality of spaced apart features attached to or projected into the base article. The plurality of features each have at least one microscale dimension and have at least one neighboring feature having a substantially different geometry. The features are separated from each other and the average feature spacing is about 1 nanometer to about 500 micrometers. The topography is numerically representable using at least one periodic function; the periodic function being representable by a pathway situated substantially between a plurality of patterns of the spaced apart features.

In one embodiment, the first feature spacing is between 0.5 micrometers (μm) and 5 μm in at least a portion of the surface. In another embodiment, the first feature spacing is between 15 and 60 μm in at least a portion of said surface. As noted above, the periodic function comprises two different sinusoidal waves. In one embodiment, the topography resembles the topography of shark-skin (e.g., a Sharklet). In another embodiment, the pattern comprises at least one multi-element plateau layer disposed on a portion of the surface, wherein a spacing distance between elements of the plateau layer provides a second feature spacing; the second feature spacing being substantially different when compared to said first feature spacing.

In yet another embodiment, an article having a surface topography for resisting bioadhesion of organisms, comprises a base article having a surface. The surface has a topography that comprises a pattern defined by a plurality of spaced apart features attached to or projected into the base article. The plurality of features comprise at least one feature having a substantially different geometry. The features are separated from each other. One of these features that is a part of the pattern is shared by a neighboring pattern. The plurality of spaced apart features has at least one microscale dimension. The neighboring patterns are separated from each other by a tortuous pathway. The tortuous pathway has at least two or more directions.

In another embodiment, an article comprises a plurality of spaced features. The features are arranged in a plurality of groupings; the groupings of features comprise repeat units. The spaced features within a grouping are spaced apart at an average distance of about 0.5 to about 200 micrometers. The groupings of features are arranged with respect to one another so as to define a tortuous pathway, the groupings have patterns of features wherein one or more features are shared between groupings. These are generally referred to as shared features. The plurality of spaced feature extend outwardly from a surface.

In one embodiment, a sum of a number of features shared by two neighboring groupings is equal to an odd number. In another embodiment, a sum of a number of features shared by two neighboring groupings is equal to an even number.

In one embodiment, the plurality of spaced features has a similar chemical composition to the surface. In another embodiment, the plurality of spaced features has a different chemical composition from that of the surface. In one embodiment, the features have similar geometries, while in another embodiment, the features can have different geometries. As will be detailed below, the groupings show dilational symmetry.

The micropatterns micropatterns have been shown to inhibit the migration and colonization of microbes in both laboratory settings and animal models. The micropattern can be incorporated onto the surface of the IUS and/or the attached monofilament string during manufacturing. The micropattern on the IUS device will reduce migration of microbes into the uterus, that cause dangerous and life-altering infections.

IUSs are primarily small devices that are implanted into the uterus of women. The IUS has a monofilament string that is affixed to the distal end of the device and is used for device removal. The pattern would be added to the device primarily on the monofilament string to reduce colonization of bacteria and migration of bacteria into the uterus, while the IUS proper would have the Sharklet micropattern to reduce both microbial colonization as well as general biofouling of the device from various bodily fluids.

Intrauterine devices (IUDs) precede the current IUS. IUDs incorporated anti-microbial copper in the past to effect both antibacterial and spermicidal properties. Antimicrobial copper is expensive to deploy in medical devices such as the IUDs. Antimicrobial copper has also been shown to lack effectiveness immediately following exposure to bacteria, which could result in migration into the uterus. Copper is a kill technology for cells, which could increase bacterial resistance in the long term, worsening the ongoing problem healthcare professionals have with resistant strains of bacteria. Copper has an associated toxicity that is a drawback for use in the body. Additionally, the addition of copper can potentially increase menstrual bleeding (menorrhagia) as well as cramping (dysmenorrhea).

Previous IUSs had smooth or inconsequential textures on the surface of the device. One embodiment of the proposed IUS would cover the device itself and the attached monofilament with the disclosed micropattern. In another embodiment, the Sharklet micropattern could be applied to a flexible sheath that covers the length of the monofilament that passes through the cervix.

The plurality of spaced features is applied to the surface in the form of a coating and can comprise an organic polymer, a ceramic or a metal. As noted above, the groupings of features are arranged with respect to one another so as to define a linear pathway or a plurality of channels. The tortuous pathway is defined by a sinusoidal function.

In one embodiment, the features are periodic. In another embodiment, the features are aperiodic. The features have a roughness factor (R) of about 2 to about 20.

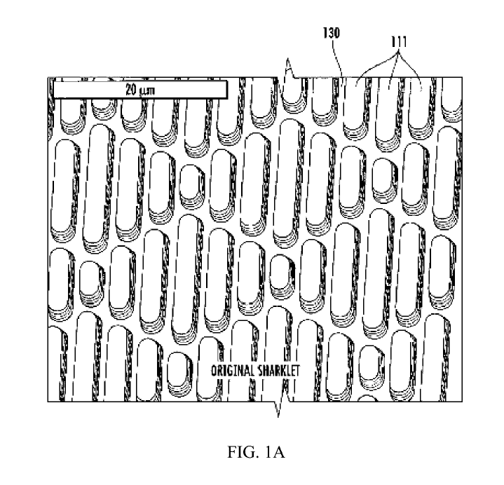

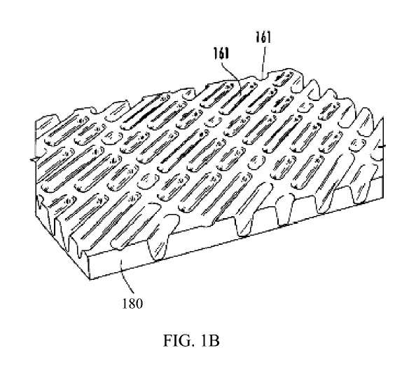

As can be seen in the FIGS. 1(A), 1(B), 2(A), 2(B), 2(C), 2(D), 5, 5(A), 7(A), 7(B) and 7(C), the article comprises a plurality of spaced features; the spaced features being arranged in a plurality of groupings. The FIGS. 1(A), 1(B), 2(A), 2(B), 2(C), 2(D), 5 (A), 5(B), 7(A), 7(B) and 7(C) show that the groupings of features comprise at least some repeat units. As can be seen in these Figures, the groupings have patterns of features. As can also be seen in these figures, the groupings of features are arranged with respect to one another so as to define a tortuous pathway when viewed in one direction and define linear pathways when viewed in other directions.

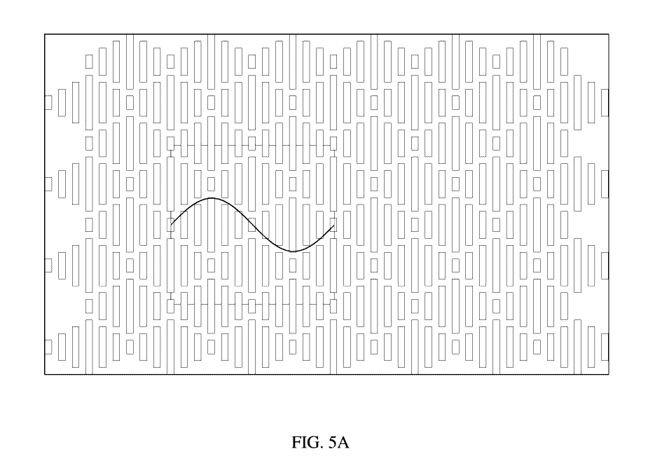

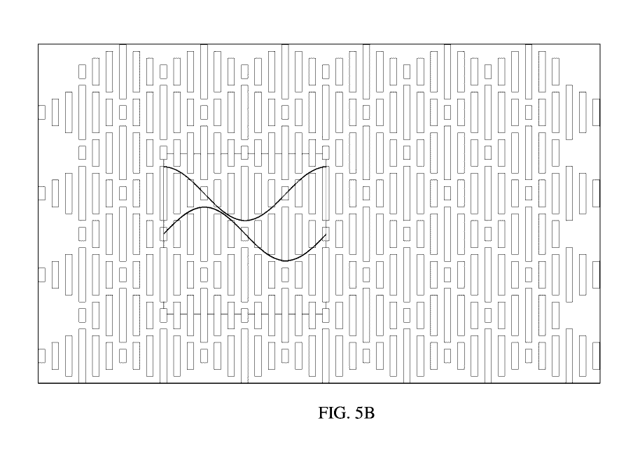

As can be seen in the FIGS. 5(A) and 5(B), the tortuous pathway exists substantially between pluralities of groupings of such features. As can be seen in the FIG. 5(A), an occasional feature may lie in the otherwise tortuous pathway. In one embodiment, a tangent to the tortuous pathway will always intersect a single separated feature of the pattern. In one embodiment, a frequency of intersection between the tangent to the tortuous pathway and the spaced feature is periodic. In another embodiment, a frequency of intersection between a tangent to the tortuous pathway and a spaced feature is aperiodic. In another embodiment, a frequency of intersection between a tangent to the tortuous pathway and a shared feature is periodic. In another embodiment, a frequency of intersection between a tangent to the tortuous pathway and the shared spaced feature is aperiodic.

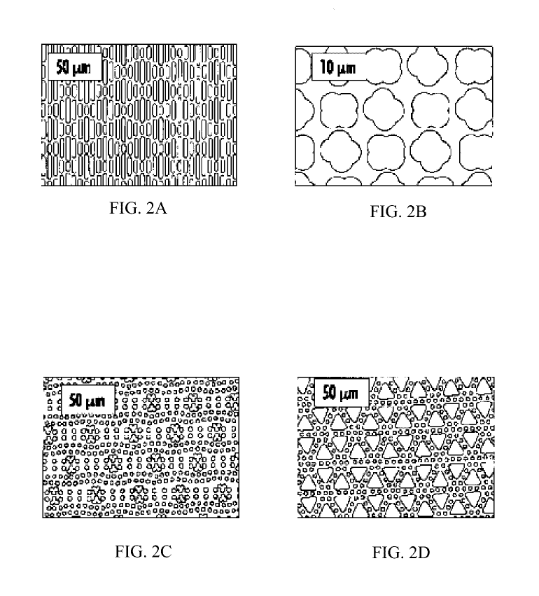

It is generally desirable for the groupings of features to comprise at least one repeat unit and to share at least one common feature. For example, in the FIGS. 1(A), 1(B), the groupings of feature have a repeat unit that has a diamond shape. It can also be seen that the smallest feature in each repeat unit is shared by two adjacent repeat units or by two adjacent groups of features. The sharing of the feature by two or more groups of patterns results in the formation of the tortuous pathway. Similarly the FIGS. 2(A) and 2(B) show at least one feature that is shared by two adjacent repeat units.

The number of features in a given pattern can be odd or even. In one embodiment, if the total number of features in a given pattern are equal to an odd number, then the number of shared features are generally equal to an odd number. In another embodiment, if the total number of features in a given pattern are equal to an even number, then the number of features in the given pattern are equal to an even number.

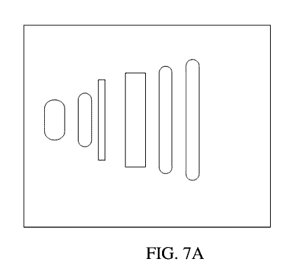

The spaced features can have variety of geometries and can exist in one, two or three dimensions or any dimensions therebetween. The spaced features can have similar geometries with different dimensions or can have different geometries with different dimensions. For example, in the FIG. 1(A), the spaced features are of a similar shape, with each shape having a different sizes, while in the FIGS. 7(A), 7(B) and 7(C), the spaced features have different geometries and different dimensions.

The geometries can be regular (e.g., described by Euclidean mathematics) or irregular (e.g., described by non-Euclidean mathematics). Euclidean mathematics describes those structures whose mass is directly proportional to a characteristic dimension of the spaced feature raised to an integer power (e.g., a first power, a second power or a third power). In one embodiment, the geometries can comprise shapes that are described by Euclidean mathematics such as, for example, lines, triangles, circles, quadrilaterals, polygons, spheres, cubes, fullerenes, or combinations of such geometries.

For example, the FIGS. 1(A) and 1(B) show that the spaced features are almost elliptical, i.e., the cross-sectional geometry of each feature when viewed from the top-down is similar to that which could be obtained by combining rectangles with semi-circles. Similarly, the FIGS. 2(B), 2(C) and 2(D) show features that comprise circles, sections of circles (e.g., semi-circles, quarter-circles), triangles, and the like.

In one embodiment, a repeat unit can be combined with a neighboring repeat unit so as to produce a combination of spaced apart features that have a geometry that is described by Euclidean mathematics. As can be seen in the FIGS. 2(C) and 2(D), the respective repeat units can be combined to produce different geometries. For example in the FIG. 2(D), the repeat unit can be combined with a single neighboring repeat unit to produce a diamond shaped geometry. Similarly, 3 or more neighboring repeat units can be combined to produce a rhombohedral, while six repeat units can be combined to produce a hexagon. Thus repeat units may be combined to produce structures whose geometries can be described by Euclidean mathematics.

In one embodiment, the spaced features can have irregular geometries that can be described by non-Euclidean mathematics. Non-Euclidean mathematics is generally used to describe those structures whose mass is directly proportional to a characteristic dimension of the spaced feature raised to a fractional power (e.g., fractional powers such as 1.34, 2.75, 3.53, or the like). Examples of geometries that can be described by non-Euclidean mathematics include fractals and other irregularly shaped spaced features.

In one embodiment, spaced features whose geometries can be described by Euclidean mathematics may be combined to produce features whose geometries can be described by non-Euclidean mathematics. In other words, the groupings of features can have dilational symmetry. The fractal dimension can be measured perpendicular to the surface upon which the features are disposed or may be measured parallel to the surface upon which the features are disposed. The fractal dimensions are measured in the inter-topographical gaps.

In one embodiment, the fractal dimensions can have fractional powers of about 1.00 to about 3.00, specifically about 1.25 to about 2.25, more specifically about 1.35 to about 1.85 in a plane measured parallel to the surface upon which the features are disposed. In another embodiment, the fractal dimensions can have fractional powers of about 1.00 to about 3.00, specifically about 1.25 to about 2.25, more specifically about 1.35 to about 1.85 in a plane measured perpendicular to the surface upon which the features are disposed.

In yet another embodiment, the fractal dimensions can have fractional powers of about 3.00 to about 4.00, specifically about 3.25 to about 3.95, more specifically about 3.35 to about 3.85 in a plane measured perpendicular to the surface upon which the features are disposed. In other words, the tortuous pathway or the surface of each feature may be textured with features similar to those of the pattern (albeit on a smaller scale), thus creating micro-tortuous pathways and nano-tortuous pathways within the tortuous pathway itself.

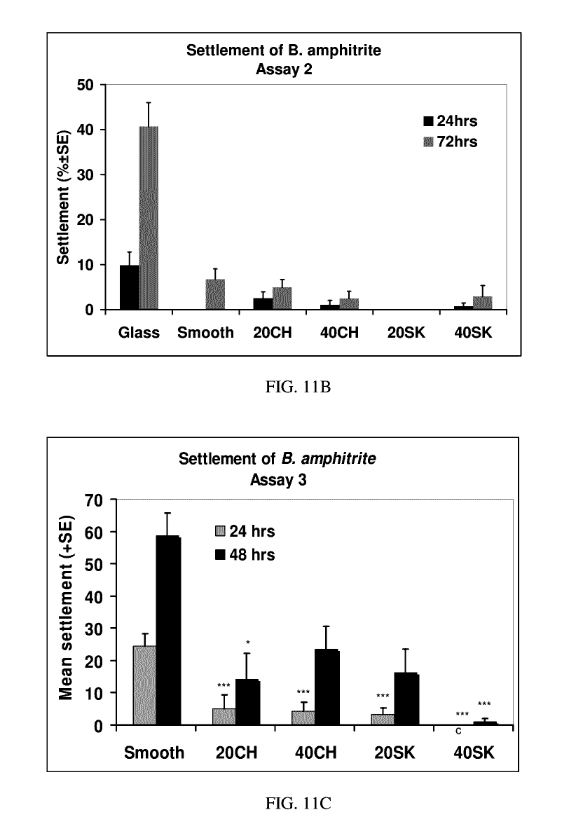

In another embodiment, the spaced features may have multiple fractal dimensions in a direction parallel to the surface upon which the features are disposed. The spaced features may be arranged to have 2 or more fractal dimensions, specifically 3 or more dimensions, specifically 4 or more dimensions in a direction parallel to the surface upon which the features are disposed. As can be seen in the FIG. 12 (A), the features have 3 different fractal dimensions in a plane parallel to the surface upon which the features are disposed. The fractal dimensions created by the features in a direction from the top to the bottom of the micrograph are 1.444 and 1.519 respectively, while the fractal dimension created by the features in a direction from left to right have dimensions of 1.557. The presence of the texture having multiple fractal dimensions prevents bioadhesion of algae, bacteria, virus, and other organisms.

In yet another embodiment, the spaced features may have multiple fractal dimensions in a direction perpendicular to the surface upon which the features are disposed. The spaced features may be arranged to have 2 or more fractal dimensions, specifically 3 or more dimensions, specifically 4 or more dimensions in a direction parallel to the surface upon which the features are disposed.

As will be noted below, the tortuous pathway may be defined by a sinusoidal function, a spline function, a polynomial function, or the like. The tortuous pathway generally exists between a plurality of groupings of spaced features and may occasionally be interrupted by the existence of a feature or by contact between two features. For example, in the FIGS. 5(A) and 5(B), the sinusoidal tortuous pathway intersects with the commonly shared feature and is thereby interrupted by it. The frequency of the intersection between the tortuous pathway and the spaced feature may be periodic or aperiodic. In one embodiment, the tortuous pathway may have a periodicity to it. In another embodiment, the tortuous pathway may be aperiodic. In one embodiment, two or more separate tortuous pathways never intersect one another.

The tortuous pathway can have a length that extends over the entire length of the surface upon which the pattern is disposed, if the features that act as obstructions in the tortuous pathway are by-passed. The width of the tortuous pathway as measured between two adjacent features of two adjacent patterns are about 10 nanometers to about 500 micrometers, specifically about 20 nanometers to about 300 micrometers, specifically about 50 nanometers to about 100 micrometers, and more specifically about 100 nanometers to about 10 micrometers.

The spaced features have linear pathways or channels between them. In one embodiment, the spaced features can have a plurality of linear pathways or a plurality of channels between them.

The spaced features can be periodic or aperiodic. As can be seen in the FIG. 1(A), the spaced features can be periodic, while as seen in the FIGS. 7(A), 7(B) and 7(C), the spaced features can be aperiodic. In a similar manner, the patterns can be periodic or aperiodic.

As noted above, the spaced features can have different dimensions (sizes). The average size of the spaced features can be nanoscale (e.g., they can be less than 100 nanometers) or greater than or equal to about 100 nanometers. In one embodiment, the spaced features can have average dimensions of 1 nanometer to 500 micrometers, specifically about 10 nanometers to about 200 micrometers, and more specifically about 50 nanometers to about 100 micrometers.

In another embodiment, the average periodicity between the spaced features can be about 1 nanometer to about 500 micrometers. In one embodiment, the periodicity between the spaced features can be about 2, 5, 10, 20, 50, 100 or 200 nanometers. In another embodiment, the average periodicity between the spaced features can be about 2, 5, 10, 20, 50, 100 or 200 nanometers. In another embodiment, the periodicity can be about 0.1, 0.2, 0.5, 1, 5, 10, 20, 50, 100, 200, 300, 400 or 450 micrometers. In yet another embodiment, the average periodicity can be about 0.1, 0.2, 0.5, 1, 5, 10, 20, 50, 100, 200, 300, 400 or 450 micrometers.

In one embodiment, the spaced features can have dimensions of 1 nanometer to 500 micrometers, specifically about 10 nanometers to about 200 micrometers, and more specifically about 50 nanometers to about 100 micrometers.

In another embodiment, the periodicity between the spaced features can be about 1 nanometer to about 500 micrometers. In one embodiment, the periodicity between the spaced features can be up to about 2, 5, 10, 20, 50, 100 or 200 nanometers. In another embodiment, the periodicity between the spaced features can be about 2, 5, 10, 20, 50, 100 or 200 nanometers. In another embodiment, the periodicity can be up to about 0.1, 0.2, 0.5, 1, 5, 10, 20, 50, 100, 200, 300, 400 or 450 micrometers. In yet another embodiment, the periodicity can be up to about 0.1, 0.2, 0.5, 1, 5, 10, 20, 50, 100, 200, 300, 400 or 450 micrometers.

In one embodiment, each feature of a pattern has at least one neighboring feature that has a different geometry (e.g., size or shape). A feature of a pattern is a single element. Each feature of a pattern has at least 2, 3, 4, 5, or 6 neighboring features that have a different geometry from the feature. In one embodiment, there are at least 2 or more different features that form the pattern. In another embodiment, there are at least 3 or more different features that form the pattern. In yet another embodiment, there are at least 4 or more different features that form the pattern. In yet another embodiment, there are at least 5 or more different features that form the pattern.

In another embodiment, at least two identical features of the pattern have at least one neighboring feature that has a different geometry (e.g., size or shape). A feature of a pattern is a single element. In one embodiment, two identical features of the pattern have at least 2, 3, 4, 5, or 6 neighboring features that have a different geometry from the identical features. In another embodiment, three identical features of the pattern have at least 2, 3, 4, 5, or 6 neighboring features that have a different geometry from the identical features.

In another embodiment, each pattern has at least one or more neighboring patterns that have a different size or shape. In other words, a first pattern can have a second neighboring pattern that while comprising the same features as the first pattern can have a different shape from the first pattern. In yet another embodiment, each pattern has at least two or more neighboring patterns that have a different size or shape. In yet another embodiment, each pattern has at least three or more neighboring patterns that have a different size or shape. In yet another embodiment, each pattern has at least four or more neighboring patterns that have a different size or shape.

As noted above the chemical composition of the spaced features can be different from the surface. The spaced features and the surfaces from which these features are projected or projected into can also comprise organic polymers or inorganic materials.

Organic polymers used in the spaced features and/or the surface can be may be selected from a wide variety of thermoplastic polymers, blend of thermoplastic polymers, thermosetting polymers, or blends of thermoplastic polymers with thermosetting polymers. The organic polymer may also be a blend of polymers, copolymers, terpolymers, or combinations comprising at least one of the foregoing organic polymers. The organic polymer can also be an oligomer, a homopolymer, a copolymer, a block copolymer, an alternating block copolymer, a random polymer, a random copolymer, a random block copolymer, a graft copolymer, a star block copolymer, a dendrimer, a polyelectrolyte (polymers that have some repeat groups that contain electrolytes), a polyampholyte (a polyelectrolyte having both cationic and anionic repeat groups), an ionomer, or the like, or a combination comprising at last one of the foregoing organic polymers.

Examples of the organic polymers are polyacetals, polyolefins, polyacrylics, polycarbonates, polystyrenes, polyesters, polyamides, polyamideimides, polyarylates, polyarylsulfones, polyethersulfones, polyphenylene sulfides, polyvinyl chlorides, polysulfones, polyimides, polyetherimides, polytetrafluoroethylenes, polyetherketones, polyether etherketones, polyether ketone ketones, polybenzoxazoles, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, polythioesters, polysulfones, polysulfonamides, polyureas, polyphosphazenes, polysilazanes, styrene acrylonitrile, acrylonitrile-butadiene-styrene (ABS), polyethylene terephthalate, polybutylene terephthalate, polyurethane, ethylene propylene diene rubber (EPR), polytetrafluoroethylene, perfluoroelastomers, fluorinated ethylene propylene, perfluoroalkoxyethylene, polychlorotrifluoroethylene, polyvinylidene fluoride, polysiloxanes, or the like, or a combination comprising at least one of the foregoing organic polymers.

Examples of polyelectrolytes are polystyrene sulfonic acid, polyacrylic acid, pectin, carageenan, alginates, carboxymethylcellulose, polyvinylpyrrolidone, or the like, or a combination comprising at least one of the foregoing polyelectrolytes.

Examples of thermosetting polymers suitable for use in the polymeric composition include epoxy polymers, unsaturated polyester polymers, polyimide polymers, bismaleimide polymers, bismaleimide triazine polymers, cyanate ester polymers, vinyl polymers, benzoxazine polymers, benzocyclobutene polymers, acrylics, alkyds, phenol-formaldehyde polymers, novolacs, resoles, melamine-formaldehyde polymers, urea-formaldehyde polymers, hydroxymethylfurans, isocyanates, diallyl phthalate, triallyl cyanurate, triallyl isocyanurate, unsaturated polyesterimides, or the like, or a combination comprising at least one of the foregoing thermosetting polymers.

Examples of blends of thermoplastic polymers include acrylonitrile-butadiene-styrene/nylon, polycarbonate/acrylonitrile-butadiene-styrene, acrylonitrile butadiene styrene/polyvinyl chloride, polyphenylene ether/polystyrene, polyphenylene ether/nylon, polysulfone/acrylonitrile-butadiene-styrene, polycarbonate/thermoplastic urethane, polycarbonate/polyethylene terephthalate, polycarbonate/polybutylene terephthalate, thermoplastic elastomer alloys, nylon/elastomers, polyester/elastomers, polyethylene terephthalate/polybutylene terephthalate, acetal/elastomer, styrene-maleicanhydride/acrylonitrile-butadiene-styrene, polyether etherketone/polyethersulfone, polyether etherketone/polyetherimide polyethylene/nylon, polyethylene/polyacetal, or the like.

Polymers that can be used for the pattern or the substrate include biodegradable materials. Suitable examples of biodegradable polymers are as polylactic-glycolic acid (PLGA), poly-caprolactone (PCL), copolymers of polylactic-glycolic acid and poly-caprolactone (PCL-PLGA copolymer), polyhydroxy-butyrate-valerate (PHBV), polyorthoester (POE), polyethylene oxide-butylene terephthalate (PEO-PBTP), poly-D,L-lactic acid-p-dioxanone-polyethylene glycol block copolymer (PLA-DX-PEG), or the like, or combinations comprising at least one of the foregoing biodegradable polymers. The biodegradable polymers upon undergoing degradation can be consumed by the body without any undesirable side effects.

In one embodiment, the pattern can comprise a polymeric resin that is blended with a biologically active agent to form a drug coating. The biologically active agent is then gradually released from the pattern, which simply acts as a carrier. When the polymeric resin is physically blended (i.e., not covalently bonded) with the biologically active agent, the release of the biologically active agent from the drug coating is diffusion controlled. It is generally desirable for the pattern to comprise an amount of about 5 weight percent (wt %) to about 90 wt % of the biologically active agent based on the total weight of the drug coating. Within this range, it is generally desirable to have the biologically active agent present in an amount of greater than or equal to about 10, preferably greater than or equal to about 20, and more preferably greater than or equal to about 30 wt % based on the total weight of the drug coating. Within this range it is generally desirable to have the biologically active agent present in an amount of less than or equal to about 75, preferably less than or equal to about 70, and more preferably less than or equal to about 65 wt % based on the total weight of the drug coating. The drug coating may be optionally coated with an additional surface coating if desired. When an additional surface coating is used, the release of the biologically active agent is interfacially controlled. The drug coating may be disposed only on the surface of the features or alternatively on the surface of the tortuous pathway.

In another exemplary embodiment, the biologically active agent may be covalently bonded with a biodegradable polymer to form the drug coating. The rate of release is then controlled by the rate of degradation of the biodegradable polymer. Suitable examples of biodegradable polymers are provided above. Within this range, it is generally desirable to have the biologically active agent present in an amount of greater than or equal to about 10, preferably greater than or equal to about 20, and more preferably greater than or equal to about 30 wt % based on the total weight of the drug coating. Within this range, it is also generally desirable to have the biologically active agent present in an amount of less than or equal to about 75, preferably less than or equal to about 70, and more preferably less than or equal to about 65 wt %, based on the total weight of the drug coating.

When the pattern is used in a medical device, the drug coating may be coated onto the medical device in a variety of ways. In one embodiment, the drug coating may be dissolved in a solvent such as water, acetone, alcohols such ethanol, isopropanol, methanol, toluene, dimethylformamide, dimethylacetamide, hexane, and the like, and coated onto the medical device in the form of the pattern. In another embodiment, a monomer may be covalently bonded with the biologically active agent and then polymerized to form the drug coating, which is then applied onto the medical device in the form of the pattern. In yet another embodiment, the polymeric resin may first be applied as a coating (in the form of the pattern) onto the medical device, following which the coated device is immersed into the biologically active agent, thus permitting diffusion into the coating to form the drug coating.

In one embodiment, a biologically active agent can be added to the pattern. Te biologically active agent can be disposed upon the surface of the pattern or can be included in the pattern (e.g., mixed with the material forming the pattern). It may also be desirable to have two or more biologically active agents dispersed in a single drug coating layer. Alternatively, it may be desirable to have two or more layers of the drug coating coated upon the medical device. Various methods of coating may be employed to coat the medical device such as spin coating, electrostatic painting, dip-coating, painting with a brush, and the like, and combinations comprising at least one of the foregoing methods of coating.

Various types of biologically active agents may be used in the drug coating, which is used to coat the medical device. The coatings on the medical device may be used to deliver therapeutic and pharmaceutically biologically active agents including anti-analgesic agents, anti-arrhythmic agents, anti-bacterial agents, anti-cholinergic agents, anti-coagulant agents, anti-convulsant agents, anti-depressant agents, anti-diabetic agents, anti-diuretic agents, anti-fungal agents, anti-hypertensive agents, anti-inflammatory agents, anti-malarial agents, anti-neoplastic agents, anti-nootropic agents, anti-Parkinson agents, anti-retroviral agents, anti-tuberculosis agents, anti-tussive agents, anti-ulcerative agents, anti-viral agents, or the like, or a combination comprising at least one of the foregoing therapeutic and pharmaceutically biologically active agents.

Examples of other suitable therapeutic and pharmaceutically biologically active agents are anti-proliferative/antimitotic agents including natural products such as vinca alkaloids (e.g., vinblastine, vincristine, and vinorelbine), paclitaxel, epidipodophyllotoxins (e.g., etoposide, teniposide), antibiotics (e.g., dactinomycin, actinomycin D, daunorubicin, doxorubicin, penicillin V, penicillin G, ampicillin, amoxicillin, cephalosporin, tetracycline, doxycycline, minocycline, demeclocycline, erythromycin, aminoglycoside antibiotics, polypeptide antibiotics, nystatin, griseofulvin, and idarubicin), anthracyclines, mitoxantrone, bleomycins, plicamycin, mithramycin and mitomycin, enzymes (L-asparaginase, which systemically metabolizes L-asparagine and deprives cells which do not have the capacity to synthesize their own asparagine), antiplatelet agents such as G(GP) IIb/IIIa inhibitors and vitronectin receptor antagonists, anti-proliferative/antimitotic alkylating agents such as nitrogen mustards (e.g., mechlorethamine, cyclophosphamide and analogs, melphalan, chlorambucil), ethylenimines and methylmelamines (e.g., hexamethylmelamine and thiotepa), alkyl sulfonates-busulfan, nitrosoureas (e.g., carmustine (BCNU) and analogs, streptozocin), trazenes-dacarbazinine (DTIC), anti-proliferative/antimitotic antimetabolites such as folic acid analogs (e.g., methotrexate), pyrimidine analogs (e.g., fluorouracil, floxuridine, cytarabine), purine analogs and related inhibitors (e.g., mercaptopurine, thioguanine, pentostatin and 2-chlorodeoxyadenosine {cladribine}), platinum coordination complexes (e.g., cisplatin, carboplatin), procarbazine, hydroxyurea, mitotane, aminoglutethimide, hormones (e.g., estrogen), anti-coagulants (e.g., heparin, synthetic heparin salts and other inhibitors of thrombin), fibrinolytic agents (e.g., tissue plasminogen activator, streptokinase and urokinase), aspirin, dipyridamole, ticlopidine, clopidogrel, abciximab, antimigratory, antisecretory (e.g., breveldin), anti-inflammatory: such as adrenocortical steroids (e.g., cortisol, cortisone, fludrocortisone, prednisone, prednisolone, 6α-methylprednisolone, triamcinolone, betamethasone, and dexamethasone), non-steroidal agents (e.g., salicylic acid derivatives such as aspirin, para-aminophenol derivatives such as acetominophen, indole and indene acetic acids (e.g., indomethacin, sulindac, etodalac), heteroaryl acetic acids (e.g., tolmetin, diclofenac, ketorolac), arylpropionic acids (e.g., ibuprofen and derivatives), anthranilic acids (e.g., mefenamic acid, meclofenamic acid), enolic acids (e.g., piroxicam, tenoxicam, phenylbutazone, oxyphenthatrazone), nabumetone, gold compounds (e.g., auranofin, aurothioglucose, gold sodium thiomalate), immunosuppressives (e.g., cyclosporine, tacrolimus (FK-506), sirolimus (e.g., rapamycin, azathioprine, mycophenolate mofetil), angiogenic agents such as vascular endothelial growth factor (VEGF), fibroblast growth factor (FGF), angiotensin receptor blockers, nitric oxide donors, anti-sense oligionucleotides and combinations thereof, cell cycle inhibitors, mTOR inhibitors, and growth factor receptor signal transduction kinase inhibitors, retenoids, cyclin/CDK inhibitors, HMG co-enzyme reductase inhibitors (statins) or protease inhibitors.

Algaecides may also be disposed upon the pattern or blended in with the material used to form the pattern. Examples of suitable algaecides are 2,2-dibromo-3-nitrilopropionamide (DNP), methylene bis-thiocyanate (MBT), 5-chloro-2-methyl-4-isothiazolin-3-one/2-methyl-4-isothiazolin-3-one (CMI), tetrahydro-3,5-dimethyl-2H,1,3,5-thiadiazine-2-thione (TDD), sodium dimethyldithiocarbamate/sodium ethylene bis dithiocarbamate (SDT), alkyl dimethylbenzyl amonium chloride family, poly[oxyethylene (dimethyliminio) ethylene (dimethyliminio) ethylene dichloride, copper sulfate, or the like, or a combination comprising at least one of the foregoing algaecides.

In one embodiment, the biologically active agents may be encapsulated in microballoons and incorporated into the pattern as part of the drug coating. In another embodiment, the biologically active agents may be encapsulated in microballoons and incorporated on the surface of the pattern or incorporated into the channels that form the tortuous pathway. The microballoons may serve to release the drugs gradually over a period of time. In other words the pattern can serve as a time-release coating for the drugs. In one embodiment, the pattern can release drugs after being subjected to a stress.

The inorganic materials used in the spaced features and/or the surface can comprise ceramics and/or metals. The inorganic materials can comprise inorganic oxides, inorganic carbides, inorganic nitrides, inorganic hydroxides, inorganic oxides having hydroxide coatings, inorganic carbonitrides, inorganic oxynitrides, inorganic borides, inorganic borocarbides, or the like, or a combination comprising at least one of the foregoing inorganic materials. Examples of suitable inorganic materials are metal oxides, metal carbides, metal nitrides, metal hydroxides, metal oxides having hydroxide coatings, metal carbonitrides, metal oxynitrides, metal borides, metal borocarbides, or the like, or a combination comprising at least one of the foregoing inorganic materials.

Examples of suitable inorganic oxides include silica (SiO₂), alumina (Al₂O₃), titania (TiO₂), zirconia (ZrO₂), ceria (CeO₂), manganese oxide (MnO₂), zinc oxide (ZnO), iron oxides (e.g., FeO, γ-Fe₂O₃, Fe₃O₄, or the like), calcium oxide (CaO), manganese dioxide (MnO₂ and Mn₃O₄), or combinations comprising at least one of the foregoing inorganic oxides. Examples of inorganic carbides include silicon carbide (SiC), titanium carbide (TiC), tantalum carbide (TaC), tungsten carbide (WC), hafnium carbide (HfC), or the like, or a combination comprising at least one of the foregoing carbides. Examples of suitable nitrides include silicon nitrides (Si₃N₄), titanium nitride (TiN), or the like, or a combination comprising at least one of the foregoing. Examples of suitable borides are lanthanum boride (LaB₆), chromium borides (CrB and CrB₂), molybdenum borides (MoB₂, Mo₂B₅ and MoB), tungsten boride (W₂B₅), or the like, or combinations comprising at least one of the foregoing borides. Exemplary inorganic substrates are those that comprise naturally occurring or synthetically prepared silica and/or alumina. Metals used in the spaced features and/or the surface can be transition metals, alkali metals, alkaline earth metals, rare earth metals, or the like, or a combination comprising at least one of the foregoing metals. Examples of metals are iron, copper, aluminum, tin, tungsten, chromium, gold, silver, titanium, or a combination comprising at least one of the foregoing metals.

The pattern can be aligned such that linear channels between respective features in a pattern can be arranged to be perpendicular and/or parallel to an average direction of fluid flow, when the pattern is disposed on a surface that contacts a flowing fluid. In one embodiment, the pattern may be disposed on the substrate so that the linear channels between respective features in a pattern can be arranged to occupy an angle of 1 to about 360 degrees, specifically about 5 to about 270 degrees, and more specifically about 10 to about 200 degrees, and more specifically about 20 to about 200 degrees with respect to the average direction of fluid flow.

In one embodiment, the pattern can be dynamically modified during use. In other words, the surface can comprise a material such as, for example, a shape memory alloy, a shape memory polymer, a magnetorheological fluid, an electrorheological fluid, and the like, that can be activated when desired to either inhibit or to facilitate bioadhesion.

In an exemplary embodiment, when the pattern comprises an organic polymer, the organic polymer can be filled with an electrically conducting filler thereby making the surface electrically conductive. By passing an electrical signal to the surface, the pattern can be heated and consequently the dimensions of the features and those of the patterns can be changed during use.

Although not required to practice the present invention, Applicants not seeking to be bound by the mechanism believed to be operable to explain the efficacy of the present invention, provide the following. The efficacy of surfaces according to the invention is likely to be due to physically interfering with the settlement and adhesion of microorganisms, such as algae, bacteria and barnacles. Properly spaced features (such as “ribs”) formed on or formed in the surface can be effective for organisms from small bacteria (<1 μm, such as 200 to 500 nm), to large tube worms (>200 μm, such as 200 to 500 μm), provided the feature spacing scales with the organism size. Specifically, bioadhesion is retarded when the specific width of closely packed, yet dissimilar features (e.g. ribs) in the pattern is too narrow to support settlement on top, yet the ribs are too closely packed to allow settlement in between. However, a feature spacing too small is believed to make the surface look flat to the settling organism, i.e. like the base surface, and thus ineffective. Accordingly, a feature spacing that scales with 25 to 75% of the settling organism's smallest physical dimension has been found to be generally effective to resist bioadhesion. Various different surface topographies can be combined into a hierarchical multi-level surface structure to provide a plurality of spacing dimensions to deter the settlement and adhesion of multiple organisms having multiple and wide ranging sizes simultaneously, such as algae, spores and barnacles.

Topographies according to the invention can generally be applied to a wide variety of surfaces for a wide variety of desired applications. Applications for inhibiting bioadhesion using the invention described in more detail below include base articles used in marine environments or biomedical or other applications which may be exposed to contamination by biological organisms, such as roofs on buildings, water inlet pipes in power plants, catheters, cosmetic implants, and heart valves. As described below, surfaces according to the invention can be formed on a variety of devices and over large areas, if required by the application.

Barnacles are known to be generally elliptically shaped have a nominal length of about 100 μm, and a nominal width of about 30 μm, Algae are also generally elliptically shaped and have a nominal length of about 7 μm, and a nominal width of about 2 μm, while spores are generally elliptically shaped have a nominal length of about 5 μm, and a nominal width of about 1.5 μm. Features according to the invention are generally raised surfaces (volumes) which emerge from a base level to provide a first feature spacing, or in the case of hierarchical multi-level surface structures according to the invention also include the a second feature spacing being the spacing distance between neighboring plateaus, which themselves preferably include raised features thereon or features projected into the base article.

As noted above, if the feature spacing is smaller than the smaller dimension of the organism or cell, it has been found that the growth is generally retarded, such between 0.25 and 0.75 of the smaller dimension of the cell or organism. A feature spacing of about ½ the smaller dimension of a given organism to be repelled has been found to be near optimum. For example, for an algae spore 2 to 5 μm in width, to retard adhesion, a feature spacing of from about 0.5 to 3.75 μm, preferably 0.75 to 2 μm is used. For example, to repel barnacles 20 to 50 μm in width, a feature spacing of between 5 and 200 μm, preferably 10 to 100 μm, has been found to be effective. For repelling both barnacles and spores, a hierarchical multi-level surface structure according to the invention can include a raised surfaces (volumes) which emerge from or are projected into a base level having a feature spacing of about 2 μm, and a plurality of striped plateau regions spaced 20 μm apart, the plateau regions also including raised surfaces (volumes) which emerge from or are projected into the plateau having a spacing of about 2 μm. One or more additional plateau regions can be used to repel additional organisms having other sizes. The additional plateau regions can be aligned (parallel) with the first plateau, or oriented at various other angles.

Although generally described for deterring bioadhesion, the invention can also be used to encourage bioadhesion, such as for bone growth. Feature dimensions of at least equal to about the size of the larger dimension of bioorganism or cells to be attached have been found to be effective for this purpose.

Although the surface is generally described herein as being an entirely polymeric, the coating can include non-polymeric elements that contribute to the viscoelastic and topographical properties. A “feature” as used herein is defined a volume (L, W and H) that projects out the base plane of the base material or an indented volume (L, W and H) which projects into the base material. The claimed architecture is not limited to any specific length. For example, two ridges of an infinite length parallel to one another would define a channel in between. In contrast, by reducing the overall lengths of the ridges one can form individual pillars. Although the surface is generally described as a coating, which is generally a different material as compared to the base article, as noted above, the invention includes embodiments where the coating and base layer are formed from the same material, such as provided by a monolithic design, which can be obtainable by micromolding.

In the case of a surface coating, the coating can comprise a non-electrically conductive material, defined as having an electrical conductivity of less than 1×10⁻⁶ S/cm at room temperature. The coating layer can comprise elastomers, rubbers, polyurethanes and polysulfones. The elastic modulus of the coating layer can be between 10 kPa and 10 MPa. In the case of 10 to 100 kPa materials, the coating can comprise hydrogels such as polyacrylic acid and thermo sensitive hydrogels such as poly isopropylacrylamide. The coating layer can be various thicknesses, such as 1 μm to 10 mm, preferably being between 100 μm to 1 mm.

Each of the features have at least one microscale dimension. In some embodiments, the top surface of the features are generally substantially planar. Although feature spacing has been found to be the most important design parameter, feature dimensions can also be significant. In a preferred embodiment of the invention, each of the features includes at least one neighboring feature having a “substantially different geometry”. “Substantially different geometry” refers to at least one dimension being at least 10%, more preferably 50% and most preferably at least 100% larger than the smaller comparative dimension. The feature length or width is generally used to provide the substantial difference.

The feature spacing in a given pattern should generally be consistent. Studies by the present Inventors have indicated that small variations in micrometer scale spacing of the ribs that compose the surface features have demonstrated that less than 1 μm changes (10% or less than the nominal spacing) can significantly degrade coating performance. For example, the consistency of a 2 μm nominal spacing should be within ±0.2 μm for best retardation of Ulva settlement.

The composition of the patterned coating layer may also provide surface elastic properties, which also can provide some bioadhesion control. In a preferred embodiment when bioadhesion is desired to be minimized, the coated surface distributes stress to several surrounding features when stress is applied to one of the features by an organism to be repelled from the surface.

The roughness factor (R) is a measure of surface roughness. R is defined herein as the ratio of actual surface area (R_{act}) to the geometric surface area (R_{geo}); R=R_{act}/R_{geo}). An example is provided for a 1 cm² piece of material. If the sample is completely flat, the actual surface area and geometric surface area would both be 1 cm². However if the flat surface was roughened by patterning, such as using photolithography and selective etching, the resulting actual surface area becomes much greater that the original geometric surface area due to the additional surface area provided by the sidewalls of the features generated. For example, if by roughening the exposed surface area becomes twice the surface area of the original flat surface, the R value would thus be 2.

The typography generally provides a roughness factor (R) of at least 2. It is believed that the effectiveness of a patterned coating according to the invention will improve with increasing pattern roughness above an R value of about 2, and then likely level off upon reaching some higher value of R. In a preferred embodiment, the roughness factor (R) is at least 4, such as 5, 6, 7, 8, 9, 10 11, 12, 13, 14, 15, 16, 17, 18, 19, 20, 25 or 30. Assuming deeper and more closely spaced features can be provided, R values can be higher than 30.

FIG. 1(A) is a scanned SEM image of an exemplary “Sharklet” topography according to an embodiment of the invention sized to resist algae adhesion and growth. The Sharklet topography is based on the topography of a shark's skin. Shark skin is a prominent example of a low friction surface in water. Unlike real shark skin which has fixed topographical feature dimensions based on the species, the Sharklet topography is scalable to any topographical feature dimension including feature width, feature height, feature geometry, and spacing between features. The composition of real shark skin is limited to the natural composition of the skin. The Sharklet topography according to the invention can be produced in a variety of material including synthetic polymers, ceramics, and metals, as well as composites.

The Sharklet and related topographies according to the invention can be described quantitatively using two sinusoidal functions. This description is provided below.

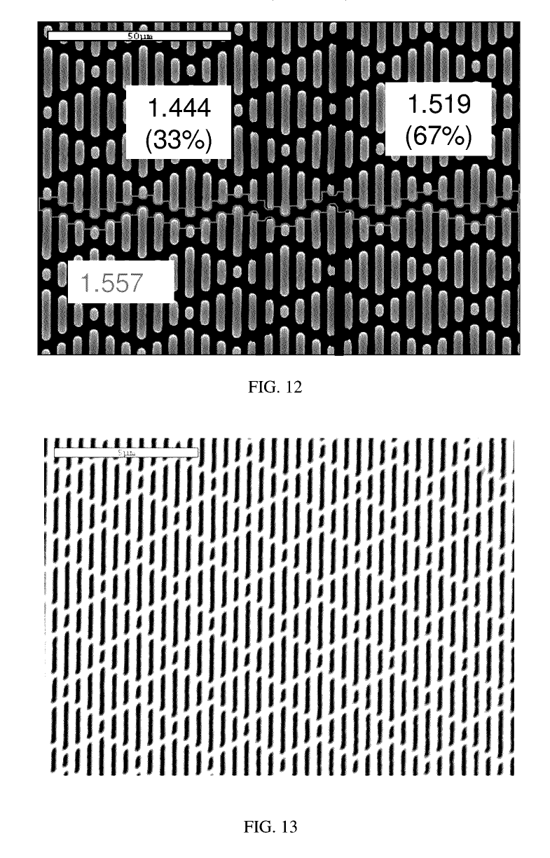

Surface layer comprises a plurality of features 111 which are attached to and project out from base surface 130. Base surface 130 can be a roofing material, the inner surface of a water inlet pipe for a power or water treatment plant, an implantable medical device or material, such as a breast implant, a catheter or a heart valve. Each of the features 111 have at least one microscale dimension, with a width of about 3 μm, lengths of from about 3 to about 16 μm, and a feature spacing of about 1.5 μm. The thickness (height) of features 111 comprising coating layer is about three (3) microns. The first feature spacing can be less than or equal to about 200 nanometers as can be seen in the FIG. 13.

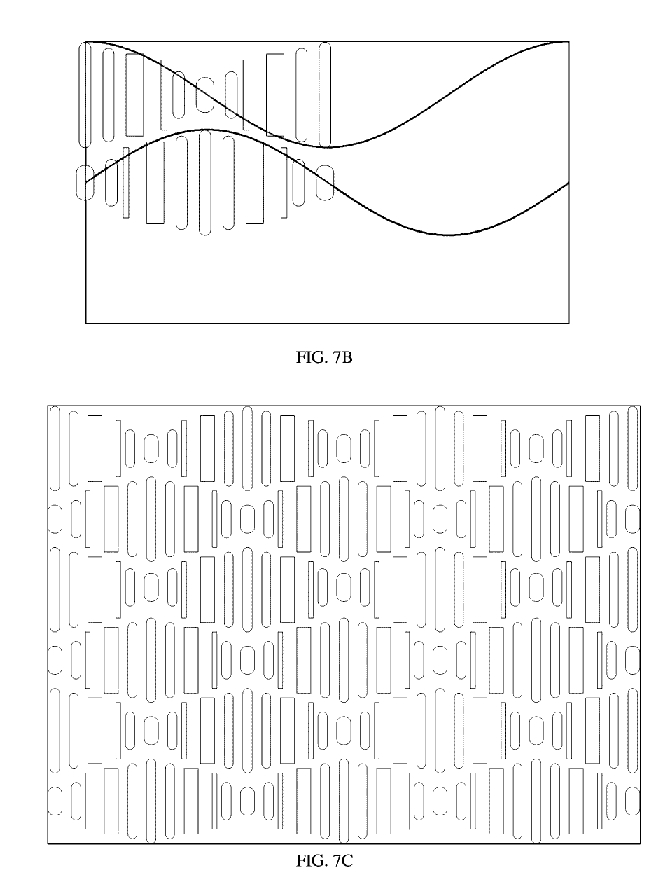

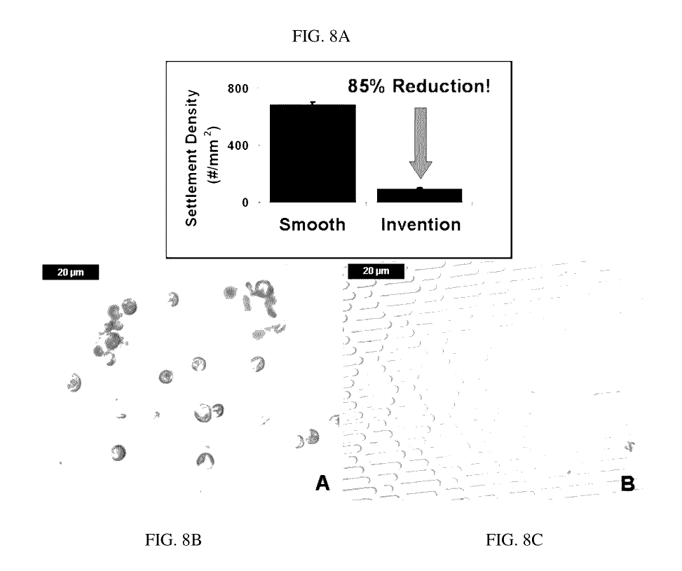

Features adjacent to a given feature 111 generally provide substantially different dimensions, in the arrangement shown in FIG. 1(A), feature lengths. The top surface of the features is shown as being planar. The patterned coating layer generally resists algae as compared to a generally planar base surface as described in the Examples and shown in FIGS. 8(A)-(C).

FIG. 1(B) is a scanned optical profilometry image of a pattern having a plurality of features 161 projecting into a base surface 180, according to another embodiment of the invention. Features 161 comprise indented void volumes into base surface 180. Although not shown, a surface can include regions having raised features 111 shown in FIG. 1(A) together with regions having indented features 161 shown in FIG. 1(B).

The composition of the patterned surface shown in FIGS. 1(A) and 1(B) is generally a polymer such as polymethylsiloxane (PDMS) elastomer SILASIC T2® provided by Dow Corning Corp, which is an elastomer of a relative low elastic modulus. The features 111 need not be formed from a single polymer. Features can be formed from copolymers and polymer composites. In another embodiment, the surface or coating comprises of a material such as, steel or aluminum, or a ceramic. The coating layer is also typically hydrophobic, but can also be neutral or hydrophilic.

As noted above, the patterned surface layer may also provide surface elastic properties which can influence the degree of bioadhesion directly, an in some cases, also modulate surface chemistry of the surface layer. It is believed that a low elastic modulus of the patterned coating layer tends to retard bioadhesion, while a high elastic modulus tends to promote bioadhesion. A low elastic modulus is generally from about 10 kPa and 10 MPa, while a high elastic modulus is generally at least 1 GPa.

The patterned surface can be formed or applied using a number of techniques, which generally depend on the area to be covered. For small area polymer layer applications, such as on the order of square millimeters, or less, techniques such as conventional photolithography, wet and dry etching, and ink-jet printing can be used to form a desired polymer pattern. When larger area layers are required, such as on the order of square centimeters, or more, spray, dipcoat, hand paint or a variant of the well known “applique” method be used. These larger area techniques would effectively join a plurality of smaller regions configured as described above to provide a polymer pattern over a large area region, such as the region near and beneath the waterline of a ship.

A paper by Xia et al entitled “Soft Lithography” discloses a variety of techniques that may be suitable for forming comparatively large area surfaces according to the invention. Xia et al. is incorporated by reference into the present application. These techniques include microcontact printing, replica molding, microtransfer molding, micromolding in capillaries, and solvent-assisted micromolding, which can all generally be used to apply or form topographies according to the invention to surfaces. This surface topography according to the invention can thus be applied to devices as either a printed patterned, adhesive coating containing the topography, or applied directly to the surface of the device through micromolding.

Another tool that can be used is the Anvik HexScan® 1010 SDE microlithography system which is a commercially available system manufactured by Anvik Corporation, Hawthorne, N.Y. 10532. Such a tool could be used to produce surface topographies according to the invention over a large area very quickly. It has a 1 micron resolution which can produce our smallest pattern at a speed of approximately 90 panels (10″ by 14″) per hour.

FIGS. 2(A)-(D) illustrate some exemplary architectural patterns (unit cells) that can be used with the invention. FIG. 2(A) shows a riblet pattern fabricated from PDMS elastomer having features spaced about 2 μm apart on a silicon wafer. The features were formed using conventional photolithographic processing. FIG. 2(B) shows a star/clover pattern, FIG. 2(C) a gradient pattern, while FIG. 2(D) shows a triangle/circle pattern.

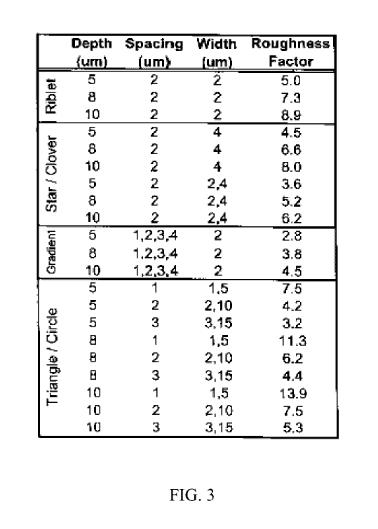

FIG. 3 provides a table of exemplary feature depths, feature spacings, feature widths and the resulting roughness factor (R) based on the patterns shown in FIGS. 2(A)-(D). Regarding the riblet pattern shown in FIG. 2(A) for the depth, spacing and widths shown, the resulting pattern roughness factor (R) ranged from 5.0 to 8.9. Similar data for the star/clover pattern (FIG. 2(B)), gradient pattern (FIG. 2(C)), and triangle/circle (FIG. 2(D)) are also shown in FIG. 3. Regarding the triangle/circle arrangement (FIG. 2(D)), for a feature depth of 10 feature spacing of 1 and feature width of 1 μm (circles) and 5 μm (triangles), a roughness factor (R) of 13.9 is obtained.

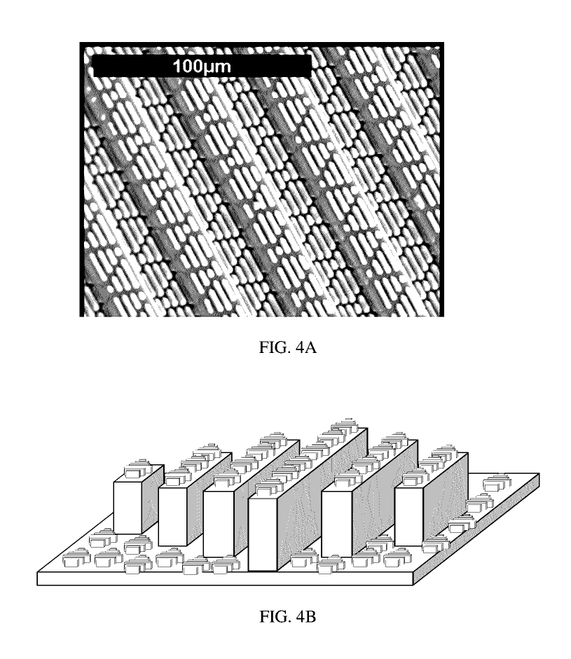

FIG. 4(A) is a scanned SEM image of an exemplary hierarchical (multi-layer) surface architecture according to an embodiment of the invention. The first feature spacing distance of about 2 μm between features 412 and its neighboring features including feature 411 is for deterring a first organism, or organism in a size range of about 5 μm, or less. For example, as noted above, an algae spore is nominally 5 μm wide. A patterned second layer comprising a plurality of striped plateau regions 420 is disposed on the first layer. A spacing distance between elements of the plateau layer provides a second feature spacing, which is substantially different as compared to the first feature spacing. As used herein, a “substantially different spacing distance” is at least 50% larger, and is preferably at least 100% larger than the smaller first feature spacing distance. In FIG. 4, the architecture shown provides a spacing distance between the second pattern strips of about 20 or about 900% greater than the first spacing distance. The 20 μm spacing is approximate ½ the width (smallest dimension) of a nominal barnacle thus repelling barnacles. Thus, hierarchical (multi-layer) surface architectures according to the invention can simultaneously repel multiple organisms covering a significant range of sizes.

In one embodiment, the hierarchical surface architecture comprises a first plurality of spaced features upon which are disposed a second plurality of spaced features. FIG. 4(B) depicts a hierarchical surface architecture comprising a first plurality of spaced features upon which are disposed a second plurality of spaced features. The spaced features of the first plurality of spaced features arranged in a plurality of groupings. The groupings of the first plurality of spaced features comprise repeat units. The spaced features within a grouping being spaced apart at an average distance of about 1 nanometer to about 500 micrometers. Each feature may have a surface that is substantially parallel to a surface on a neighboring feature. In one embodiment, each feature may have a surface that is not parallel to a surface on a neighboring feature. Each feature is separated from the neighboring features. The groupings of features are arranged with respect to one another so as to define a tortuous pathway; the plurality of spaced features provide the article with an engineered roughness index of about 5 to about 30.

The second plurality of spaced features are disposed upon the first plurality of spaced features. The spaced features of the second plurality of spaced features are arranged in a plurality of groupings. The groupings of features of the second plurality of spaced features comprise repeat units. The spaced features within a grouping being spaced apart at an average distance of about 1 nanometer to about 50 micrometers; specifically at an average distance of about 10 nanometer to about 30 micrometers; and more specifically at an average distance of about 20 nanometer to about 10 micrometers. The groupings of features in the second plurality of spaced features are arranged with respect to one another so as to define a tortuous pathway; the plurality of spaced features providing the article with an engineered roughness index of about 5 to about 30. The second plurality of spaced features may have disposed thereon a third plurality of spaced features, and so on.

In one embodiment of the invention, the surface topography is a topography that can be numerically represented using at least one sinusoidal function. In the paragraphs below, a sinusoidal description of Sharklet and related topographies is provided. The Sharklet and related topographies can be numerically representing using two (2) sinusoidal waves. A general equation is provided which the only topographical restriction is that two elements with at least a one dimensional length discrepancy must be selected and periodic throughout the structure. The smallest feature of the two being related to the size of the smallest dimension (the width) of the organism of interest. All the elements and features in-between and/or around the two periodic features become irrelevant. Examples of each of these instances are presented and the generalized equation is then developed.

The Sharklet shown in FIG. 1(A) will be used for this example. The dimensions are not relevant as this point. The Sharklet shown in FIG. 1(A) is a 4-C element (repeating) structure.

FIG. 5(A) shows a sinusoidal wave beginning at the centroid of the smallest of the four Sharklet features. By inspection of the periodicity of the Sharklet features, a sine wave of the form y=A sin (wx) can be used to describe this periodicity as shown in FIG. 5(A). It can be noticed that the repeating structure above the section described by the sine wave is out of phase from that structure by 90 degrees or π/2 radians, which happens to be a cosine wave. That periodicity and packing can be represented using a cosine wave in the form y=B+A cos (wx) (as shown in FIG. 5(B)).

The entire surface area of the topography can be numerically represented by a numerical summation of both sinusoidal waves in the form:

y=cN+A sin(wx)

y=cN+B+A cos(wx) where N=0,1,2,3 . . . n

The area of coverage of the topography is thus described by the limits of n and x.

The Sharklet and related topographies can thus be defined by the following limitations:

(i) Two geometric features of at least one dimensional discrepancy must be periodic throughout the structure.
(ii) The smallest of the two geometric features is related to the smallest dimension of the fouling organism or cell of interest.
(iii) In a standard Cartesian coordinate system represented by x and y, with the origin positioned at start of each sin and cosine wave, the smaller of the two features is periodic where the waves cross y=0. The waves pass through the area centroid of the feature @ y=0.
(iv) In a standard Cartesian coordinate system represented by x and y with the origin positioned at start of each sine and cosine wave, the larger of the two features is periodic where the waves cross reaches it's maximum amplitude. The wave intersects the center of the tallest part of the feature @ y=max and the x-moment of inertia of the feature @ y=0. General Form of Sinusoids

y=cN+A sin(wx)

y=cN+B+A cos(wx)
where N=0, 1, 2, 3 . . . n
   The following equations define the values for the variables A, B, c and w:

A=(½)*(L_{D})
L_{D}=y-dimension of larger of two elements

B=(½)*(S_{D})+(P_{S})+(½)*(L_{D})
S_{D} y-dimension of smaller of two elements
P_{S}=y-spacing between the two elements after packing
c=L_{D}+2*(P_{S})+S_{D}
w=2πf=(2π)/(T)→w: angular frequency (rad), f: frequency (Hz), T: wave period

T=2*X_{D}
X_{D}=x-dimensions from centroid of smaller feature to the center of the tallest point on the larger feature

### Example Units=Microns

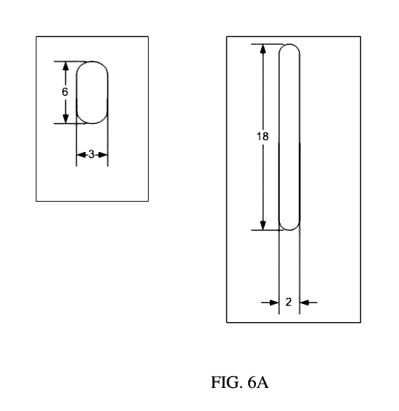

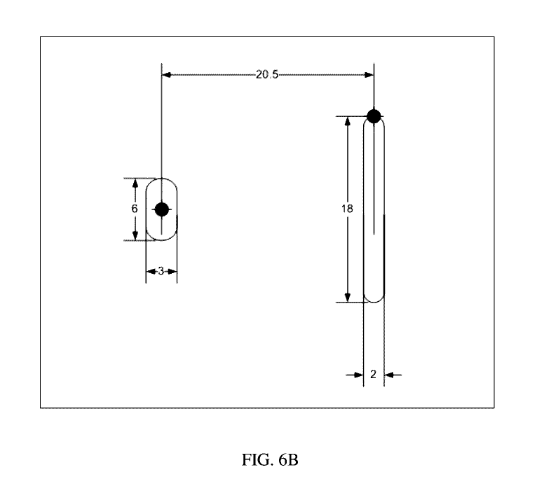

FIG. 6(A) shows element 1 and element 2. FIG. 6(A) shows that the two elements can have different sizes. In the FIG. 6(A), the element 1 has a shorter length and a greater width than the element 2. FIG. 6(B) shows the resulting layout after following limitations 3 & 4 and defining X_{D}. The FIG. 6(B) depicts that the spacing between elements can be varied.

Variables are then calculated:

A=(½)*(18)=9

B=(½)*(6)±(3)±(½)*(18)=15

c=18+(2)*(3)+9=33w=(2π)/(2*20.5)=π/20.5

Sinusoids are then defined.

y=33N+9 sin((π/20.5)x) (1)

y=33N+15+9 cos((π/20.5)x) (2)

N=0, 1, 2, 3 . . . n

The space is then filled with elements between defined elements as shown in FIG. 7(A). Sinusoidal waves are then applied to define periodic repeat definitions as shown in FIG. 7(B) to create the desired topographical structure over the desired surface area shown in FIG. 7(C).

Another method for describing surface topographies according to the invention involves a newly devised engineered roughness index (ERI), first conceived of and used by the present Inventors. The ERI can characterize the roughness of an engineered surface topography. The ERI was developed to provide a more comprehensive quantitative description of engineered surface topography that expands on Wenzel's roughness factor (Wenzel R N. 1936, Resistance to solid surfaces to wetting by water. Ind Eng Chem 28:988-944). It has been found that Wenzel's description alone does not adequately capture the tortuosity of the engineered topographies studied. ERI is expressed as follows in the Equation (3):

ERI=(r*df)/f_{D} (3)

wherein the ERI encompasses three variables associated with the size, geometry, and spatial arrangement of the topographical features: Wenzel's roughness factor (r), depressed surface fraction (f_{D}), and degree of freedom for movement (d_{f}).

Wenzel's roughness factor refers to the ratio of the actual surface area to the projected planar surface area. The actual surface area includes areas associated with feature tops, feature walls, and depressed areas between features. The projected planar surface area includes just the feature tops and depressions.

The depressed surface fraction (f_{D}) is the ratio of the recessed surface area between protruded features and the projected planar surface area. This depressed surface fraction term is equivalent to both 1-φ_{S} and 1-f₁ where φ_{S} is the surface solid fraction as described by Quéré and colleagues (Bico J, Thiele U, Quéré D. 2002. Wetting of textured surfaces. Colloids Surf A: Physicochem Eng Aspects 206:41-46; Quéré D. 2002. Rough ideas on wetting. Physica A: Stat Theoret Phys 313:32-46) and f₁ is the solid-liquid interface term of the Cassie-Baxter relationship for wetting (Cassie A B D, Baxter S. 1944. Wettability of porous surfaces, Trans Faraday Soc 40:546-551).

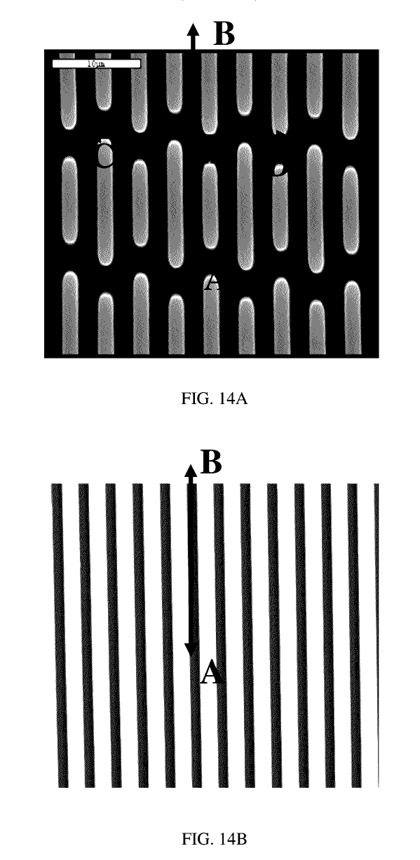

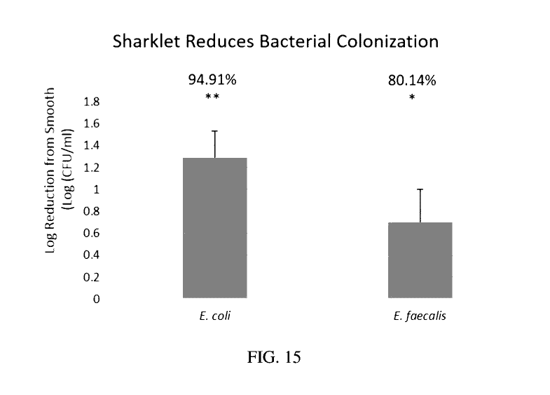

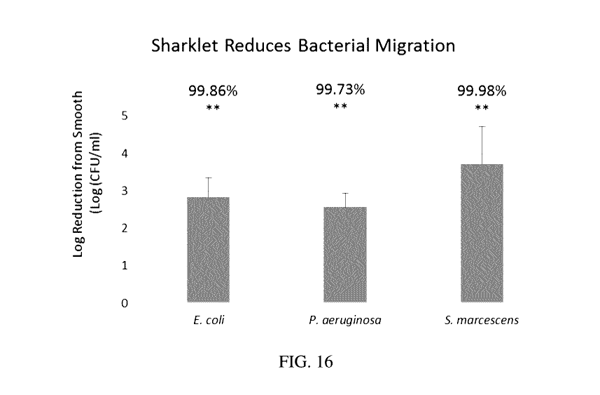

The degree of freedom for movement relates to the tortuosity of the surface and refers to the ability of an organism (e.g. Ulva spore or barnacle) to follow recesses (i.e., grooves) between features within the topographical surface. If the recesses form a continuous and intersecting grid, movement in both the x and y coordinates on a plane surface is permitted and the degree of freedom will be 2. Alternatively, if the grooves are individually isolated (e.g. as in channel topographies) then movement is only allowed in one coordinate direction, the degree of freedom will be 1. The calculation of degrees of freedom can be seen in the FIG. 14. FIG. 14A contains a pattern where an organism traveling in a channel between two raised features can, upon arriving at the point X migrate in either the direction A-B or the direction C-D. It therefore has two degrees of freedom available to it. In the FIG. 14B, an organism travelling can only travel in the direction A-B along the channel. The organism therefore has only one degree of freedom.

In addition to being able to control bioadhesion, it is desirable for the surface to function as a non-wetting surface to any fluid. This is accomplished by minimizing the value a f_{D} in the Equation (3) above. In minimizing the ERI value, the value of r/f_{D} is increased, thereby increasing the ERI value.

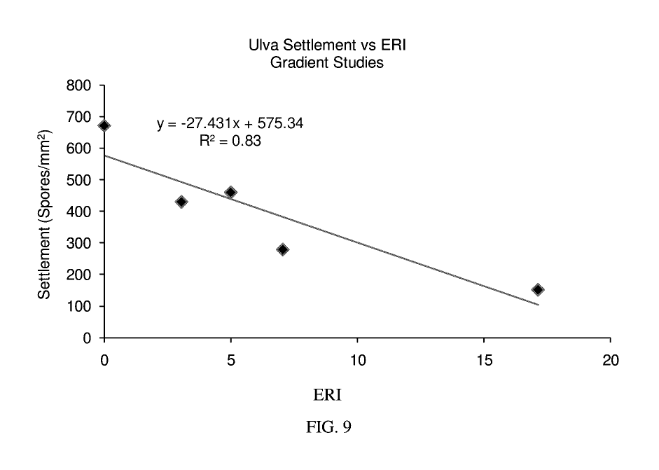

As such, as shown in FIG. 9 described in detail below, larger ERI values correlate with reduced settlement. It is generally desirable to have ERI values of about 5 to about 40, specifically about 7 to about 30, and more specifically about 9 to about 20. In a preferred embodiment, the ERI is at least 5, preferably 8 or more, preferably 10 or more, preferably 15 or more.

A related surface description according to another embodiment of the invention comprises a polymer layer having a surface. The polymer layer is an elastomer containing a plurality of dissimilar neighboring protruding non-planar surface features where for repelling algae, the features are spaced between 0.5 and 5.0 microns. The features are such that the stress required to bend the feature is 10% greater than the stress required to strain a cell wall and where the features have a greater than 10% bending modulus difference in the bending modulus between two neighboring features, or in the case of three or more neighboring features, their vector equivalence difference of greater than 10%. Preferably, the surface features exist on the surface at a features per area concentration of greater than 0.1 square microns.

The invention provides numerous benefits to a variety of applications since surface properties can be customized for specific applications. The invention can provide reduced energy and cost required to clean surfaces of biofouling by reducing biofouling in the first place. As a result, there can be longer times between maintenance/cleaning of surfaces. As explained below, the invention can also provide non-capsule formation due to foreign body response in the case of coated implanted articles. The invention can also be configured to provide enhanced adhesion to surfaces.

The present invention is thus expected to have broad application for a variety of products. Exemplary products that can benefit from the bioadhesion resistance provided by coating architectures according to the invention include, but are not limited to, the following:
a. biomedical implants, such as breast plant shells or other fluid filled implant shells;
b. biomedical instruments, such as heart valves;
c. Hospital surfaces, e.g., consider film (electrostatic) applications to surfaces that can be readily replaced between surgeries;
d. Clothing/protective personal wear;
e. Biomedical packaging;
f Clean room surfaces, such as for the semiconductor or biomedical industry;
g. Food industry, including for packaging, food preparation surfaces;
h. Marine industry-including exterior surfaces of marine vessels including ships and associated bilge tanks and gray water tanks and water inlet/outlet pipes;
i. Water treatment plants including pumping stations;
j. Power plants;
k. Airline industry;
l. Furniture industry, such as for children's cribs;
m. Transportation industry, such as for ambulances, buses, public transit, and
n. Swimming pools

The articles may find utility in biomedical implants, such as breast plant shells or other fluid filled implant shells; biomedical instruments, such as heart valves; hospital surfaces (e.g., consider film (electrostatic) applications to surfaces that can be readily replaced between surgeries); clothing/protective personal wear; biomedical packaging, such as, for example, the outside surface of sterilized packaging; clean room surfaces, such as, for example, the semiconductor or biomedical industry; food industry, such as, for example, food packaging, food preparation surfaces; marine industry, such as, for example, exterior surfaces of marine vessels including ships and associated bilge tanks, gray water tanks and water inlet/outlet pipes; water treatment plants, such as, for example, pumping stations; power plants; airline industry; furniture industry, such as, for examples, for children's cribs, handles on exercise equipment, and exercise equipment; in the transportation industry, such as, for example, in ambulances, buses, public transit; swimming pools and other structures that are used in aquatic environments; and the like. Additional details of the types of articles and surfaces upon which the pattern can be disposed are provided below.

The pattern may be used in articles that include medical devices, medical implants, medical instruments that are used internal or external to the body of living beings. The term “living beings” can include warm blooded animals, cold blooded animals, trees, plants, mammals, fishes, reptiles, amphibians, crustaceans, and the like. The medical devices, medical implants and medical instruments may be temporarily or permanently inserted into the body of the living being. Examples of medical devices, medical implants and medical instruments are endotracheal tubes; stents; shells used to encapsulate implants such as, for example, breast implant shells; breast implants; ear tubes; heart valves; surfaces of bone implants; surfaces of grafted tissues; surfaces of contact lens; components and surfaces of dialysis management devices such as, for example, a dialysis line; components and surfaces of urinary management devices such as, for example, a urinary catheter; components and surfaces of central venous devices such as, for example, a urinary catheter; surfaces of implanted devices such as, for example, pacemakers, artificial pancreas, and the like; ports on catheters such as, for example, feeding tube ports, implanted venous access ports. It is to be noted that the patterns can be varied to permit bioadhesion or to resist bioadhesion. These variations include geometrical variations, dimensional variations, variations in surface chemistry, or the like. These variations may be static or dynamic variations.

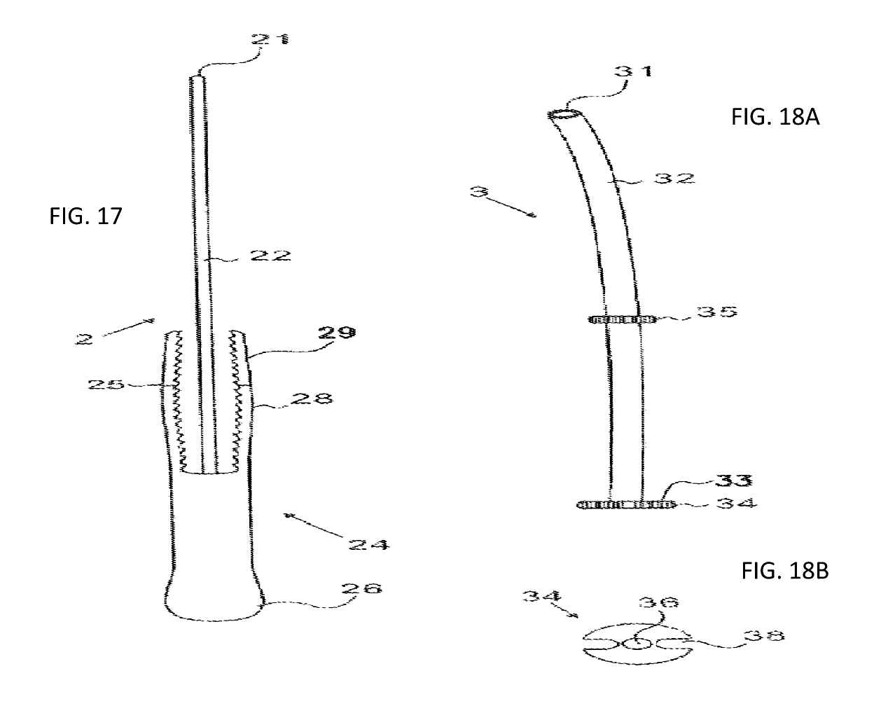

As noted above, the pattern may be disposed on an intrauterine device. Referring to the drawings, FIGS. 17 and 18 depict the disassembled parts of an inserter for an intrauterine device in accordance with an embodiment of the invention. Inserter 10 comprises a sleeve part 3 and a plunger part 2. Parts 2 and 3 can be easily assembled manually with no need for any tools and similarly can be easily disassembled into two separate parts to facilitate the removal of the inserter after the intrauterine device is positioned correctly inside the uterus

Plunger part 2 comprises a thin rod 22 and a handle 24 fixedly attached to one end of the rod. Handle 24 comprises an elongated portion 26 to facilitate gripping by hand and two opposite resilient arms 28, second engaging member 29, extending upwardly in the direction of and on opposite sides of rod 22. Arms 28 are configured as tweezers arms that can bend inwardly toward each other. Rod 22 and handle 24 are preferably made from a rigid sterilizable polymeric material such as polyethylene or the like. Any of the other polymers or blends listed above mayt be used for the various parts of the interuterine device. Rod 22, due to its small diameter/length ratio can easily flex to assume any curvature. Preferably part 2 is formed as a one integral piece, for example by mould injection. Alternatively, rod 22 can be attached to handle 24 by any other method including heat fusing, adhering and the like, or can be inserted into a bore drilled or otherwise formed in handle 24.

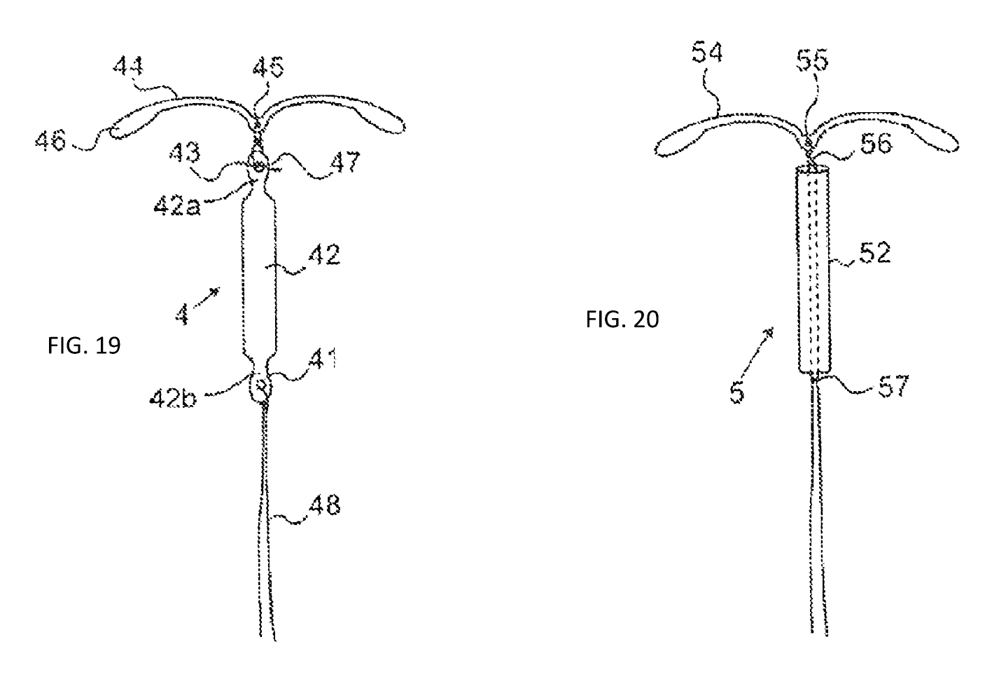

Sleeve part 3 comprises a tube 32 provided with a disk 34, first engaging member 33, at its proximal end. Tube 32 is a hollow tube made of medical grade sterilizable semi-rigid polymeric material such as polyethylene, polypropylene and the like. The inner diameter of the tube is dimensioned to receive the contraceptive body of a T-shaped intrauterine device, such as the devices depicted in FIGS. 19 and 20. The distal (upper) part of tube 32 is slightly curved to better adapt to the curvature of the uterus. The tube may be further bent before insertion and after examining the curvature of the uterus. Tube 32 may be further provided with a movable marking ring 35 which can slide along the tube. Ring 35 serves to mark the correct depth of insertion, i.e., the length of the uterine lumen, measured prior to insertion, as is well known in the art. Tube 32 may also include imprinted scaling marks (not shown) to facilitate positioning of ring 35. As shown in FIG. 18B, disk 34 comprises a central opening 36, substantially of the same diameter as the inner diameter of tube 32 and two opposite recesses 38. Recesses 38 are dimensioned to receive arms 28 of plunger 2.

Inserter 10 is assembled by inserting rod 22 of plunger 2 into tube 32 through opening 36 of disk 34 and forcing arms 28 of the plunger into recesses 34 of the disk, such that the tube can slide along the arms. As mentioned above, arms 28 are resilient and act like tweezers. When no pressure is applied on the arms, sleeve 3 can slide up and down arms 28 to assume any desired relative position between plunger and sleeve. When arms 28 are pressed against disk 36, the sleeve is locked to the plunger to maintain their relative position. Thus, disk 34 and arms 28 serve as engaging members that allow not only to easily assemble/disassemble the plunger and sleeve but also to easily lock/release their relative position. The inward surface of arms 28 may be smooth or may be toothed, as depicted in FIG. 17, to enhance the grip of disk 34 by arms 28. Alternatively, recesses 38 may be each provided with a small protrusion and arms 28 may be each provided with a complementary longitudinal slot running along the inward surface thereof for serving as a rail for sliding the disk along the arms (not shown).

In use, the inserter is held by one hand with portion 26 of handle 24 resting against the palm and aims 28 held between finger and thumb near the location of disk 34. When adjustment of the relative position of the tube and the rod is required in order to withdraw the IUD into the tube or to expose it, this can be easily done by gripping disk 36 between finger and thumb and sliding it axially along arms 28 in the required direction. When the relative position between tube and rod should be kept fixed, i.e., when the IUD is inserted through the cervix, the arms are pressed by finger and thumb against recesses 38 to lock sleeve 3 onto plunger 2, thus preventing possible relative movement between tube 32 and rod 21 and maintaining the RID in its contracted configuration at the top of tube 32. Arms 28 include markings 25 which mark the correct position of disk 34 when the inserter and IUD are ready for insertion.

The pattern disclosed herein may be may be disposed on all parts of the interuterine device. It is disposed on the thin rod 22, the handle 24, the resilient arms 28, the engaging member 29, the sleeve 3, the tube 32, the disk 34, the engaging member 33, and the like.

It will be appreciated that the inserter of the present invention has the advantage of allowing the physician to manipulate both plunger and tube by one hand during the whole insertion procedure while leaving his other hand free to use other instruments or perform other operations as necessary. It will further appreciated that inserter 10 further allows for easily disengaging the plunger part from the sleeve part, thus allows withdrawal of the inserter, after the FUD is correctly placed, not as a whole unit, but by parts, namely first the plunger then the sleeve, to ensure that the monitoring string of the IUD is not entangled within the inserter and to reduce the risk of withdrawing the IUD along with the inserter.

Inserter 10 is designed for the insertion of a T-shaped IUD that comprises a cylindrical contraceptive body, i.e., copper-bearing or hormone releasing body, and a pair of foldable arms. Inserter 10 can be used for the insertion of any IUD of this type, including Nova-T and LNG-20 (Mirena®). However, in accordance with the general objective of the invention to make insertion easier while minimizing discomfort and pain, there is also provided a new IUD directed at this objective. The IUD of the invention, unlike known T-shaped frame IUDs, does not comprise a vertical stem that runs through the contraceptive body, but rather the contraceptive body is suspended from the middle node between the two transversal anus. The elimination of a central shaft allows for reducing the diameter of the contraceptive body, making insertion easier. The absence of a central relatively rigid shaft further allows for greater flexibility of the body, resulting with reduced bleeding and pain.

FIGS. 3 and 4 depict two embodiments designated 4 and 5, respectively, of an intrauterine device of the invention. Both embodiments comprise a pair of foldable resilient wings 44, 54 and a contraceptive body 42, 52 suspended from the junction zone between the two wings. Wings 44, 54 are of substantially similar shape as of those of the Nova-T and Mirena® devices. The wings are made of one resilient piece having a junction zone from which the two convex wings divert. In their expanded relaxed configuration, the wings are generally directed at opposite lateral directions substantially perpendicularly to the elongated body and having their tips 46 directed downward. Tips 46 are of substantially hemispherical shape forming a rounded leading end when the wings are pressed against each other in the contracted configuration. Preferably tips 46 are dimensioned to be slightly wider than opening 31 of tube 32 such that when the IUD is withdrawn into tube 32 they stop the IUD at the correct position and prevent it from being withdrawn deeper into the tube. The structure and flexibility of the wings insures that the device can easily adapt to the lateral dimensions of the uterus and can easily respond to uterus contractions while minimizing the pressure applied on the uterus walls. In accordance with the embodiments shown in FIGS. 3 and 4, the wings comprise an eyelet 45, 55, respectively, in the junction zone between the wings.

In the embodiment depicted FIG. 3, body 42 is provided with two extensions 42 a and 42 b at the upper and bottom ends, respectively, which comprise eyelets 43 and 41. In accordance with this embodiment, wings 44 and body 42 are connected by a flexible string 47 which is threaded first through one of eyelets 45 and 43 and is tied to form a first knot, then is threaded and tied around the second eyelet to form a second knot and loop. The ends of the string can be cut close to the knots. A second string 48 is tied to bottom eyelet 41. String 48 is used for monitoring the IUD and to facilitate its removal. Body 42, which carries the contraceptive agent, may be a reservoir of contraceptive compound such as levonorgestrel or any other suitable progesterone analog. Body 42 may comprise a core of suitable polymeric matrix impregnated with the contraceptive agent and enveloped by a permeable membrane for controlling sustained release of the contraceptive agent into the uterus cavity over a prolonged time. Alternatively, body 42 may comprise a core enveloped with copper. Extensions 42a and 42b of body 42 may be formed as part of the envelope. It will be appreciated that body 42 may contain other or additional active therapeutic agents for treating various conditions of the uterus.

In the embodiment depicted in FIG. 4, only one string 56 is used for both connecting wings 54 to body 52 and as the monitoring and removal string. In accordance with this embodiment string 56 is inserted through the body substantially along its central longitudinal axis and is tied around eyelet 55. String 56 is inserted through body 52 either through a prefabricated channel that runs from top to bottom or by forcing the string through the resilient body by means of a needle-like instrument. The two ends of the string extending from the bottom end of body 52 are tied close to the body and serve for monitoring and removing the IUD. In accordance with the embodiment shown here, both ends of the string are inserted through the body, however it will be easily realized that alternatively only one end of the string may be inserted through the body such that the two ends of the string are extending from the opposites ends of the body, one end is tied to the wings while the other end is tied on itself to form a knot close to the body and is left to serve as the removal string. Body 52 may be of the type described above in association with FIG. 3, or alternatively may comprise copper rings or copper wire surrounding the string.

All surfaces of the interuterind device such as, for example, the resilient wings 44, 54, the contraceptive body 42, 52 suspended from the junction zone between the two wings, the elongated body and having tips 46, body 52, eyelet 55, have the textured pattern disposed on its surfaces.

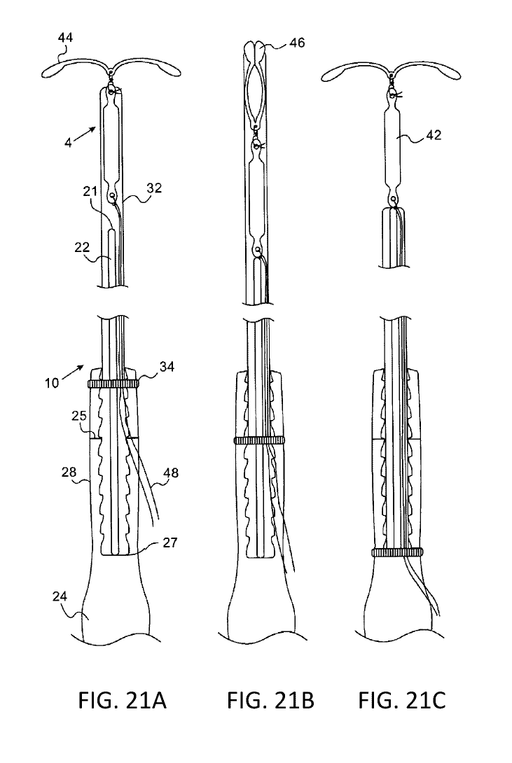

FIGS. 21A to 21C demonstrate the relative position of the tube, plunger and the IUD during storage (21A); immediately before insertion (21B); and immediately after the RID is positioned in the uterus and before the inserter in withdrawn (21C).

During storage, the intrauterine device and the inserter are kept as a kit under sterilized sealed packaging. The package is opened a short time before the insertion and only after examination to determine the size, position, and curvature of the uterus.

As depicted in FIG. 21A, the packaged kit preferably contains inserter 10 in the assembled configuration and with the contraceptive elongated body 42 of IUD 4 inside the forward end of tube 32, protected thereby. During storage, wings 44 must be stored in the expanded configuration in order to prevent fatigue which might cause the wings to lose their flexibility. If the IUD is retained in the tube with its wings folded for a prolonged period, permanent deformation might occur and the arms might not return to their expanded configuration when released from the tube. At this position, i.e., with the IUD body protected inside tube 32 and wings 44 outside tube 32, tube 32 is mounted near the free ends of arms 28 and the tip 21 of plunger rod 22 does not contact the IUD but is located at certain distance below it.

FIG. 21B demonstrates the inserter and the IUD ready for insertion. At this configuration, wings 44 are withdrawn into tube 32 to assume their contracted configuration and the tip of plunger rod 22 contacts the bottom end of the IUD. To achieve this configuration, disk 34, held between finger and thumb, is retracted on arms 28 toward the handle and/or the handle is pushed forward until disk 34 is positioned on marks 25. At this configuration the IUD is entirely housed within tube 32 but for tips 46 which are exposed, and is ready for insertion. As mentioned above, tips 46 stop the ND from being withdrawn further into the tube.

To place the IUD, the inserter as configured in FIG. 21B is inserted through the cervical canal into the uterus while pressing arms 28 inwardly against disk 34 to maintain the IUD in its contracted configuration within tube 22. When the IUD is in the correct position it is released from tube 22 by retracting disk 34 backward along arms 28 until it reaches handle 24 and cannot be further moved. FIG. 21C illustrates the configuration of the IUD and the inserter after release. The distance between marks 25 and the bottom end 27 of arms 28 substantially equals the length of the IUD in its contracted configuration.

After the IUD is correctly placed inside the uterus, inserter 10 may be withdrawn. In accordance with the invention withdrawal of the device may be performed by first withdrawing the plunger part, then withdrawing the sleeve part. Such a procedure reduces the risk that removal string 48 will get entangled between the sleeve and the plunger which might result in the IUD being displaced or accidentally removed along with the inserter. In order to remove the plunger, disk 34 may be held steady while handle 24 is pulled backward until aims 28 are released from the disk and the plunger can be removed. Thereafter the sleeve part may be withdrawn. All of the parts of the IUD shown in the FIGS. 21A-21C may have portions of the surface or the entire surface textured with the patterns disclosed herein.

### EXAMPLES

It should be understood that the Examples described below are provided for illustrative purposes only and do not in any way define the scope of the invention. An experiment was performed to compare the performance of an exemplary surface architecture according to the invention having features formed from a PDMS elastomer as compared to a planar uncoated control surface (the same PDMS elastomer) against bioadhesion of algae spores. The inventive surface topography was the Sharklet shown in FIG. 1(A). Following 45 minutes of exposure, as shown in FIG. 8(A), the settlement density of algae spores on the smooth control sample was about 720/mm², while the settlement density for the surface architecture according to the invention was only about 100/mm², or only about 15% of the settlement density of the control. FIG. 8(B) is a scanned light micrograph image of the surface of the control, while FIG. 8(C) is a scanned light micrograph image of the surface of the surface architecture according to the invention.

A further set of Ulva spore settlement assays were conducted to evaluate the impact of ERI. All pattern designs tested were transferred to photoresist-coated silicon wafers using previously described photolithographic techniques. Patterned silicon wafers were reactive ion etched, utilizing the Bosch process, to a depth of approximately 3 μm creating a topographical negative. Wafers were then stripped of photoresist and cleaned with an O₂ plasma etch. Hexamethyldisilazane was vapor deposited on the processed silicon wafers to methylate the surfaces in order to prevent adhesion. Topographical surfaces were transferred to PDMSe from replication of the patterned silicon wafers. The resultant topographies contain features projecting from the surface at a height of approximately 3 μm. Pattern fidelity was evaluated with light and scanning electron microscopy.

Ulva spore settlement assays were conducted with 76 mm×25 mm glass microscope slides coated with smooth and topographically modified PDMSe surfaces. Glass slides coated with PDMSe topographies were fabricated using a two-step curing process as previously described (Carman et al. 2006). The resultant slide (˜1 mm thickness) contained an adhered PDMSe film with a 25 mm×25 mm area containing topography bordered on both sides by 25 mm×25 mm smooth (no topography) areas.

Three replicates of each topographically-modified PDMSe sample, permanently adhered to glass microscope slides, were evaluated for settlement of Ulva spores. Topographies included the Sharklet (inset A; upper left), 2 μm diameter circular pillars (inset C; lower left), 2 μm wide ridges (inset D; lower right), and a multi-feature topography containing 10 μm equilateral triangles and 2 μm diameter circular pillars (inset B; upper right). A uniformly smooth PDMSe sample was included in the assay and served as a control for direct comparison.

Regarding the Sharklet, 2 μm ribs of various lengths were combined centered and in parallel at a feature spacing of 2 μm. The features were aligned in the following order as indicated by feature length (μm): 4, 8, 12, 16, 12, 8, and 4. This combination of features formed a diamond and was the repeat unit for the arrayed pattern. The spacing between each diamond unit was 2 μm. Similar to that of the skin of a shark in terms of feature arrangement, this pattern was designed such that no single feature is neighbored by a feature similar to itself

Regarding the 2 μm diameter circular pillars shown in inset C, patterns of 2 μm pillars and 2 μm ridges were designed at an analogous feature spacing of 2 μm. The pillars were hexagonally packed so that the distance between any two pillars was 2 μm. Regarding the 2 μm wide ridges shown in inset D, the ridges were continuous in length and spaced by 2 μm channels (D).

Regarding the multi-feature pattern shown in inset B, the pattern was designed by combining 10 μm triangles and 2 μm pillars. Pillars were arranged in the same hexagonal packing order as in the uniform structure. At periodic intervals, a 10 μm equilateral triangle replaced a set of six 2 μm pillars forming the outline of a 10 μm triangle. Thus, this design maintained a 2 μm feature spacing between each edge of the triangle and pillars.

Fertile plants of Ulva linza were collected from Wembury beach, UK (latitude 50° 18′N; 4° 02′W). Ulva zoospores were released and prepared for attachment experiments as documented previously (Callow et al. 1997).

Topographical samples were pre-soaked in nanopure water for several days prior to the assay in order for the surfaces to fully wet. Samples were transferred to artificial seawater (TROPIC MARIN®) for 1 hour prior to experimentation without exposure to air. Samples were then rapidly transferred to assay dishes to minimize any dewetting of the topographical areas. Ten ml of spore suspension (adjusted to 2×10⁶ spores per ml) were added to each dish and placed in darkness for 60 minutes. The slides were then rinsed and fixed with 2% glutaraldehyde in artificial seawater as described in Callow et al. (1997).

Spore counts were quantified using a Zeiss epifluorescence microscope attached to a Zeiss Kontron 3000 image analysis system (Callow et al. 2002). Thirty images and counts were obtained from each of three replicates at 1 mm intervals along both the vertical (15) and horizontal (15) axes of the slide.

Spore density was reported as the mean number of settled spores per mm² from 30 counts on each of three replicate slides±standard error (n=3). Statistical differences between surfaces were evaluated using a nested analysis of variance (ANOVA) followed the SNK (Student-Newman-Kuels) test for multiple comparisons. Replicate slides (3) of each surface (5) were treated as a nested variable within each surface.

The mean spore density measured for each of the studied PDMSe surfaces was plotted against the calculated engineered roughness index (ERI) to determine if any correlations existed. It must be noted that these ERI values are for a fixed feature spacing of 2 μm and depth of 3 μm.

Spores were calculated to settle at a mean density of 671±66 spores/mm² on the smooth PDMSe surface. All topographies showed a statistically significant reduction in spore density relative to this smooth surface as evaluated by ANOVA analysis followed by the SNK multiple comparison test. A lower mean spore density was measured on the triangles/pillars (279±66) compared to both the pillars (430±81) and ridges (460±54). The Sharklet topography had the lowest spore density (152±32) compared to all other surfaces.

For the 2 μm wide ridges, the majority of the settled spores were bridged between the top edges of neighboring ridges. A few smaller spores were found squeezed within the 2 μm wide channels between ridges.

Spores remained atop the hexagonal packed 2 μm diameter pillars. No settled spores were observed on flat areas between pillars. For the multi-feature topography containing both 10 μm triangles and 2 μm pillars, spores completely avoided settling on the flat top surface of the triangle. Most spores appeared to have settled on top of a pillar while leaning against the edge of the triangle feature. The table below provides the calculated EM values for the studied topographical patterns.

TABLE

| | Feature Geometry | | | Engineered Roughness Index | | | |
|---|---|---|---|---|---|---|---|
| | depth | spacing | width | | | | ERI |
| | (μm) | | | r | df | f_{D} | (r*df)/f_{D} |
| Ridges | 3 | 2 | 2 | 2.5 | 1 | 0.50 | 5 |
| Pillars | 3 | 2 | 2 | 2.36 | 2 | 0.77 | 6.1 |
| Triangles/Pillars | 3 | 2 | 2, 10 | 2.23 | 2 | 0.51 | 8.7 |
| Sharklet | 3 | 2 | 2 | 2.5 | 2 | 0.53 | 9.5 |
| Smooth | n/a | n/a | n/a | 1 | 2 | 1 | 2 |

Calculated engineered roughness index (EM) values for the studied topographical surfaces.

The mean spore density measured on each tested PDMSe surface was plotted against the calculated engineered roughness index (ERI) as shown in FIG. 9. A correlation was observed and a linear regression model was fit to the data. A fairly strong (R2=0.69, p<0.001) inverse linear relationship existed between mean spore density and ERI by the following equation:

Spore Density(spores/mm²)=796−63.5*(EM) (2).

The Sharklet had highest ERI (9.5) and lowest mean spore density. Following the trend, the triangles/pillars topography had the second highest ERI (8.7) and the second lowest mean spore density. Both the uniform ridges and pillars topographies had lower ERI values (5.0 and 6.1 respectively) and higher mean spore densities than both the Sharklet and triangles/pillars. There were no statistical differences in the mean spore densities of uniform ridges and pillars topographies.

Since feature width and spacing were the same for all these topographies, differences in ERI values were associated only with differences in feature geometry and tortuosity. This indicated that the geometric shape and arrangement of the individual features of Sharklet was likely critical because it enhanced anti-settlement effectiveness over topographies of equivalent dimensions.

Not all topographically modified or roughened surfaces have anti-settlement properties for Ulva spores. Spore settlement results on topographies presented here and previously have indicated that a critical interaction must be achieved between individual topographical features and the spore for the entire surface to be an effective inhibitory surface. Although trends with ERI values and spore settlement have been ascertained, it was only after topographic surfaces were designed at a feature spacing of 2 μm. This indicated that there exists an interaction between roughness measures and feature spacing that must be considered when designing topographic surfaces.

In another round of testing, sharklet surfaces according to the invention were prepared and tested for efficacy against barnacle adhesion. In the study, a surface comprising 20 μm×5 μm, 20 μm×20 μm, 200 μm×5 μm and 200 μm×20 μm PDMSe channels were evaluated for B. amphitrite (barnacle) settlement and release. The convention used herein is W×D, where W represents both the width and spacing between features and D represents the depth (height) of features. Although equal in this particular example, the invention is in no way limited to the width equaling the spacing. Incubation time was approximately 48 hours. All four topographies reduced settlement relative to a surface of smooth PDMSe. The most significant reduction in barnacle settlement of about 65% was provided by the 20×20 channels as shown in FIG. 10.

Based on the results of this study, surfaces were designed to probe the antifouling properties of topographical features with ˜20 μm dimensions. Four replicates each of the following PDMSe topographies were prepared: smooth, 20×20 Channels (20CH), 20×40 Channels (40CH), 20×20 Sharklet (20SK) and 20×40 Sharklet (40SK). Four 0.5 cm³ drops of artificial sea water (ASW) were deposited on each sample and then 10 cyprids dispersed in ˜0.5 cm³ ASW were added to each drop, bringing the total volume per drop to 1 cm³. Samples were next placed in a humidified incubator at 28° C. for 24 hrs. Samples were inspected and settlement numbers (cyprids attached+cyprids metamorphosed) were counted. Samples were then returned to the incubator and read after subsequent 24 hr periods following the same protocol. The results collected at 24 and 48 hrs from the initial assay are referred to in the following sections as “assay 1”.

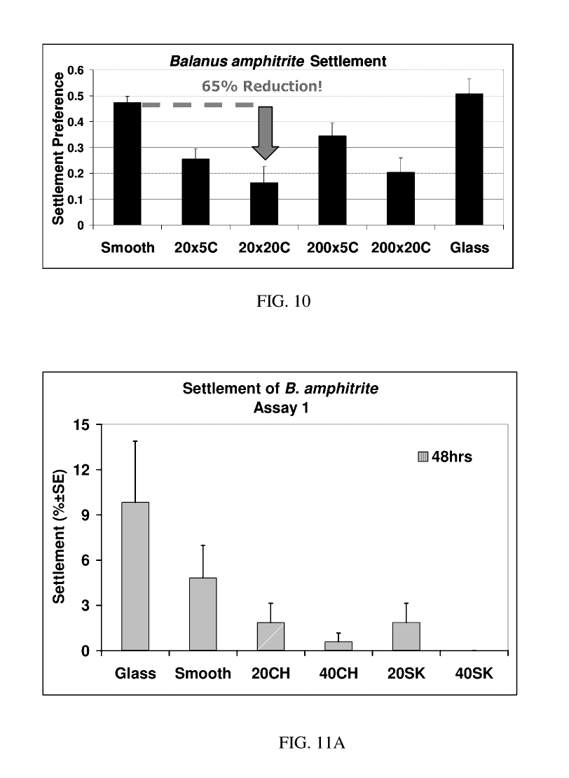

In assay 1, settlement on all surfaces was negligible after 24 hrs, but the 48 hr results shown in FIG. 11(A) appear to indicate that all of the studied topographies reduce barnacle cyprid settlement. The 40SK topography completely inhibited barnacle settlement. However, because the overall settlement counts were quite low (˜10% on glass), accurate statistical interpretation of the data was not possible.

Due to low overall cyprid settlement in assay 1, the decision was made to “clean” the test surfaces and repeat the process. Briefly, surfaces were rinsed in an excess of nanopure water, gently agitated in 90% ethanol on an orbital shaker over night, and subsequently rinsed in nanopure water and allowed to dry in air. On all PDMSe surfaces there was still a vague droplet outline evident following this procedure. Closer inspection suggested that this deposit may be bacterial biofilm. Assay 2 droplets were deposited on areas of the samples not previously used for assay. The results of this second round of testing will be referred to as “assay 2” in which readings were taken after 24 and 72 hrs. Cyprids were allowed to explore for longer in assay 2 to increase settlement, but the 48 hr reading was removed to prevent the potential loss of test droplets during transfer to and from the incubator. After the 24 hr reading, salinity in most of the test droplets had increased through evaporation from ˜33 ppt to ˜40 ppt so reverse osmosis (RO) water was added to each droplet to return the salinity back to ˜33 ppt. For assay 2, significant results were found between glass and all PDMSe surfaces as shown in

FIG. 11(B) shows barnacle settlement after both 24 and 72 hrs. The 20SK topography completely inhibited barnacle settlement. However, there were still no significant differences detected between the smooth PDMSe control and the textured inventive surfaces. A repeat experiment with a fresh cyprid batch and new test surfaces was sought so that a definitive conclusion could be reached.

After the completion of assay 2, fresh samples were used to replicate the study. Using the same protocol as before, settlement was evaluated at 24 and 48 hrs for this study (assay 3). All topographies yielded significantly lower settlement compared to smooth after 24 hrs as shown in FIG. 11(C). After 48 hrs, the 20CH and 40SK topographies both showed a significant inhibitory affect on settlement. The 40SK topography almost completely inhibited settlement of the cyprids, which is consistent with assay 1 results. No significant differences were detected between the various topographies according to the invention.

Additional tests were performed to evaluate critical surface dimensions for bacteria. Recent literature on the relationship between zoospores and bacteria cells suggest that zoospores of eukaryote alga can sense a chemical signal produced by bacteria by utilizing a bacterial sensory system. As such, bacterial biofilms have a direct influence on the development of algal communities. Work by the present Inventors with barnacle cyprids as discussed above has also shown the presence of something resembling a bacterial biofilm that was found to remain after washing. These findings suggest that disrupting the bacterial colonization of surfaces can in turn disrupt the settlement of larger organisms such as zoospores or cyprids.

In the investigation with the bacteria Staphylococcus aureus, Sharklet topography with 2 μm spacing dimensions was chosen to accommodate isolated, individual bacterium (cell size ˜1-2 μm) to prohibit connectivity between bacteria cells thus prohibiting the formation of a confluent biofilm. Samples of 2 μm Sharklet PDMSe, smooth PDMSe, and glass were statically exposed to 107 CFU/mL in growth medium for up to 12 days to promote biofilm formation. Samples were removed on the 2nd, 4th, 7th, and 12th days, gently rinsed by immersion in de-ionized water, and air-dried for characterization.

After 12 days, scanning electron micrographs (SEM) revealed abundant biofilm on glass and slightly less on the smooth PDMSe, but no evidence of biofilm on the Sharklet surface. The SEM images acquired also suggest inhibition of bacterial cell settlement on the Sharklet surface.

It is to be understood that while the invention has been described in conjunction with the preferred specific embodiments thereof, that the foregoing description as well as the examples, which follow are intended to illustrate and not limit the scope of the invention. Other aspects, advantages and modifications within the scope of the invention will be apparent to those skilled in the art to which the invention pertains.

1. An article comprising:
a first plurality of spaced features; the spaced features arranged in a plurality of groupings; the groupings of features comprising repeat units; the spaced features within a grouping being spaced apart at an average distance of about 1 nanometer to about 500 micrometers; each feature having a surface that is substantially parallel to a surface on a neighboring feature; each feature being separated from its neighboring feature; the groupings of features being arranged with respect to one another so as to define a tortuous pathway; the plurality of spaced features providing the article with an engineered roughness index of about 5 to about 30; where the article is an interuterine device.

2. The article of claim 1, wherein the plurality of spaced feature extend outwardly from a surface.

3. The article of claim 2, wherein the plurality of spaced features has a similar chemical composition to the surface.

4. The article of claim 2, wherein the plurality of spaced features has a different chemical composition from that of the surface.

5. The article of claim 2, wherein the plurality of spaced features is applied to the surface in the form of a coating.

6. The article of claim 1, wherein the plurality of spaced features comprises an organic polymer, a ceramic or a metal.

7. The article of claim 1, wherein the groupings of features are arranged with respect to one another so as to define a linear pathway or a plurality of channels.

8. The article of claim 1, wherein the tortuous pathway is defined by a sinusoidal curve.

9. The article of claim 1, wherein the tortuous pathway is defined by a spline function.

10. The article of claim 1, wherein one or more features are shared between groupings.

11. The article of claim 1, wherein the groupings have patterns of features.

12. The article of claim 1, wherein the features have similar geometries.

13. The article of claim 1, wherein the features have different geometries.

14. The article of claim 1, wherein the features have different dimensions.

15. The article of claim 1, wherein the groupings show dilational symmetry.

16. The article of claim 1, wherein the features are periodic.

17. The article of claim 1, wherein the features are aperiodic.

18. The article of claim 1, wherein the features have a depth to height ratio of about 1 to about 10 micrometers.

19. The article of claim 1, wherein the features have an average surface roughness of about 4 to about 50.

20. The article of claim 6, wherein the organic polymer is a polyacetal, a polyolefin, a polyacrylic, a polycarbonate, a polystyrene, a polyester, a polyamide, a polyamideimide, a polyarylate, a polyarylsulfone, a polyethersulfone, a polyphenylene sulfide, a polyvinyl chloride, a polysulfone, a polyimide, a polyetherimide, a polytetrafluoroethylene, a polyetherketone, a polyether etherketone, a polyether ketone ketone, a polybenzoxazole, a polyphthalide, a polyacetal, a polyanhydride, a polyvinyl ether, a polyvinyl thioether, a polyvinyl alcohol, a polyvinyl ketone, a polyvinyl halide, a polyvinyl nitrile, a polyvinyl ester, a polysulfonate, a polysulfide, a polythioester, a polysulfone, a polysulfonamide, a polyurea, a polyphosphazene, a polysilazane, a polyurethane, an ethylene propylene diene rubber, a polytetrafluoroethylene, a perfluoroelastomer, a fluorinated ethylene propylene, a perfluoroalkoxyethylene, a polychlorotrifluoroethylene, a polyvinylidene fluoride, a polysiloxane, combination comprising at least one of the foregoing organic polymers.

21. The article of claim 1, wherein the spaced features comprise an inorganic material, and wherein the inorganic material is an inorganic oxide, an inorganic carbides, an inorganic nitride, an inorganic hydroxide, an inorganic oxide having a hydroxide coating, an inorganic carbonitride, an inorganic oxynitride, an inorganic boride, an inorganic borocarbide, or a combination comprising at least one of the foregoing inorganic materials or wherein the inorganic material is a metal oxide, a metal carbide, a metal nitride, a metal hydroxide, a metal oxide having a hydroxide coating, a metal carbonitride, a metal oxynitride, a metal boride, a metal borocarbide, or a combination comprising at least one of the foregoing inorganic materials.

22. The article of claim 1, wherein the spaced features comprise a metal, the metal being iron, copper, aluminum, tin, gold, titanium, silver, tungsten, platinum, palladium, chromium, or a combination comprising at least one of the foregoing metals.

23. The article of claim 1, wherein the article has an engineered roughness index of greater than or equal to about 5.

24. The article of claim 1, where the article has an engineered roughness index of greater than or equal to about 10.

25. The article of claim 1, where the article has a drug coating disposed thereon.

26. The article of claim 1, where the article comprises biologically active agents.

27. The article of claim 1, where the article comprises a bio-degradable polymer.

28. The article of claim 1, where the article is a portion of an interuterine system.

29. The article of claim 1, where the pattern is disposed on the article by coating, injection molding, embossing, laser etching, extrusion, casting, or a combination thereof.
